(19) United States
(12) Patent Application Publication
(10) Pub. No.: US 2018/0185484 A1
(43) Pub. Date: Jul. 5, 2018

(54) PREPARATION AND USE OF PLATELET PRODUCTS

(71) Applicant: Cerus Corporation, Concord, CA (US)

(72) Inventors: William GREENMAN, San Francisco, CA (US); Lily LIN, Oakland, CA (US); Adonis STASSINOPOULOS, Dublin, CA (US)

(21) Appl. No.: 15/543,159

(22) PCT Filed: Jan. 15, 2016
(86) PCT No.: PCT/US2016/013722

§ 371 Date: Jul. 12, 2017

Related U.S. Application Data
(60) Provisional application No. 62/104,681, filed on Jan. 16, 2015.
(60) Provisional application No. 62/242,066, filed on Oct. 15, 2015.

(51) Int. Cl. A61K 41/00; A61K 35/19; A61M 1/36; A61P 7/00

(52) U.S. Cl. CPC A61K 41/0009; A61K 41/0023; A61P 7/00; A61M 1/3683; A61K 35/19

(57) ABSTRACT

The present disclosure relates to improved methods for the preparation and platelet products for administration to subjects in need thereof

### CROSS REFERENCE TO RELATED APPLICATIONS

This application claims the benefit of priority of U.S. provisional patent application Ser. No. 62/104,681, filed Jan. 16, 2015 and U.S. provisional patent application Ser. No. 62/242,066, filed Oct. 15, 2015, the contents of each of which are hereby incorporated by reference herein in their entirety.

### TECHNICAL FIELD

The methods described herein generally relate to the preparation of platelet-containing blood products. More particularly, the present disclosure relates to improved methods for the preparation and use of platelet products for administration to subjects in need thereof.

### BACKGROUND

Whole blood contains various cellular components, such as red blood cells, platelets and white blood cells, suspended in a liquid plasma component. Donations of whole blood often are separated into the individual, clinically therapeutic components of red blood cells, platelets and plasma, for individual storage and use in treating medical conditions that require administration of one or more particular blood component to a patient. Platelets play a key role in hemostasis, clot stability and retraction, as well as in vascular repair and anti-microbial host defense. Thrombocytopenia, or low blood platelet count, can result from a number of conditions, which depending on severity, may require the transfusion of donor platelets for treatment.

Any of several methods may be used to collect and store platelets during the preparation of platelet-containing products (see e.g., Vassallo et al., 2006, Curr. Opin. Hematol. 13:323-330). For example, automated cell separation systems are used in many countries to produce a platelet unit from a single donor by an apheresis procedure (plateletpheresis), while returning other non-collected blood components to the donor. Other platelet preparation methods known in the art may involve whole blood donations, from which whole-blood derived platelet units are obtained. Such whole blood-derived platelet preparation methods generally comprise more manual processes than apheresis. Some countries, including the United States, may utilize whole blood processes where platelet concentrates are obtained by isolating platelet rich plasma (PRP) from standard whole blood donations, followed by a second centrifugation to separate the platelets from remaining constituents such as plasma (PRP method). Some countries, including many European countries, use a different centrifugation process to obtain platelet concentrates from the “buffy coat” layer of donated whole blood (buffy coat or BC method). Platelet concentrates (PC) from one or more additional donors prepared using either the PRP or buffy coat methods are often pooled to generate a desired platelet product, such as a pooled platelet unit for transfusion. Generally for products comprising pooled platelets, components from four to six individual donors are combined to produce an amount or dose suitable for a single transfusion unit (e.g., therapeutic dosage unit). Such platelet products from PC are typically suspended in plasma and/or a synthetic storage solution prior to storage. Additionally, platelet preparations also may be treated with a pathogen inactivation technology to reduce the risk of potential contaminating pathogens as well as leukocytes, which is expected to increase with the size of the pool.

The presence of donor leukocytes, or T lymphocytes, in blood components intended for transfusion can have significant undesirable effects. A variety of leukocyte-related transfusion complications (e.g., leukocyte-mediated adverse reactions) have been reported, including increased risk of immune mediated-complications, such as transfusion-associated graft versus host disease (TA-GVHD), alloimmunization (e.g., HLA-alloimmunization), platelet refractoriness, febrile non-hemolytic transfusion reactions, immunosuppression and transmission or reactivation of viruses, such as cytomegalovirus. Transfused donor leukocytes may lead to complications by proliferating and destroying susceptible tissues in an immunocompromised recipient, or even when non-proliferating, by triggering immune activities through displayed antigens (e.g., HLA markers) and/or stimulation or secretion of cytokines. Therefore, it is desirable to remove leukocytes from blood components, particularly when such blood components will be used in subjects with elevated risk for leukocyte-related transfusion complications.

To reduce the likelihood of leukocyte-related complications, platelets and other blood components may be subjected to gamma or other forms of ionizing irradiation, as an established means to inactivate leukocytes for susceptible patient populations. However, gamma irradiation may not inactivate high levels of T cells and generally does not remove the inactivated leukocyte cellular material (e.g., leukocyte degradation products) or abolish the risk for leukocyte-related transfusion complications that are not dependent on leukocyte proliferation. Process leukocyte reduction steps, including apheresis collection processes and the use of various filtration media (e.g., leukofiltration devices, such as leukocyte reduction filter), also may be used to significantly reduce the amount of contaminating leukocytes. Leukofiltration is particularly common as a process step in the case of whole-blood derived platelets produced by the buffy coat and PRP methods, with the associated variability in leukocyte content, and especially when such platelet products will be provided to subjects with elevated risk for leukocyte-related transfusion complications, such as for example, stem cell and bone marrow transplant patients and oncology patients subjected to chemotherapy and/or radiation therapy. Factors such as additional cost, loss of platelet product (or subgroups of platelets) and lower performance (e.g., quality) due to manipulation are disadvantages of incorporating a leukofiltration step.

Some pathogen inactivation technologies, such as photochemical pathogen inactivation compounds, have been shown to inactivate leukocytes and have been substituted for gamma irradiation of platelet products, while used in conjunction with an additional effective leukocyte removal (e.g., leukoreduction) step, such as leukofiltration with a leukocyte reduction filter (U.S. Publication No. 2013/0131639; van Rhenen et al., 2003, Blood 101:2426-2433) or an apheresis process (Osselaer et al., 2008, Vox Sang. 94:315-323). For the psoralen-based pathogen inactivation compound amotosalen, greater than 5 logs reduction of viable T lymphocytes (e.g., inactivation of T lymphocytes) in treated platelet preparations has been achieved after photoactivation of the compound (Grass et al., 1998, Blood 91:2180-2188). This photochemical amotosalen-UVA technology has now been in routine use for more than a decade to prepare pathogen-inactivated platelets, including from whole blood using a buffy coat method, in conjunction with an additional leukofiltration step for leukocyte removal, in order to minimize risk of leukocyte-related transfusion complications (Corash et al., 2004, Bone Marrow Transplantation 33:1-7).

There remains a need for improved methods of providing whole blood-derived platelet products, such as those prepared using the buffy coat or PRP methods, which are suitable for transfusion of patients while reducing the likelihood of leukocyte-related transfusion complications. Such improved methods are particularly desirable in the case of administration of such platelet products to patients who have elevated risk for a leukocyte-related transfusion complication.

### SUMMARY

The present disclosure relates generally to improved methods for the preparation and use of platelet products for administration to subjects in need thereof, such as for example, for providing platelet products to human subjects (e.g., patients) with elevated risk for a leukocyte-related transfusion complication. Transfusion ready platelet products and related methods as described herein provide certain advantages over current methods, including for example, reducing leukocyte-related transfusion complications in a recipient of a platelet product and eliminating the need for treatment of platelets with ionizing radiation (e.g., gamma irradiation) and a filtration medium (e.g., leukocyte removal filter), together with any associated increased platelet yield and/or cost savings.

In one aspect, the present disclosure sets forth a method of providing a platelet product to a subject in need thereof, comprising administering to a human subject with elevated risk for a leukocyte-related transfusion complication a platelet product, wherein the platelet product comprises one or more platelet components (e.g., whole blood derived platelet components, platelet concentrates, platelet preparations) prepared from whole blood donation(s), and wherein the platelet product has been subjected (e.g., during preparation of the platelet product, after preparation of the platelet product) to photochemical treatment with a psoralen compound (e.g., psoralen derivative, salt thereof) to inactivate pathogens and leukocytes, if present, and wherein the platelet product has not been subjected (e.g., during preparation of the platelet product, after preparation of the platelet product) to treatment with either ionizing radiation (e.g., gamma irradiation, X-ray irradiation, X-irradiation) or a filtration medium to reduce the level of contaminating leukocytes. In some embodiments, the one or more platelet components (e.g., whole blood derived platelet components) are prepared from whole blood donation(s) by a buffy coat method. In some embodiments, the one or more platelet components (e.g., whole blood derived platelet components) are prepared from whole blood donation(s) by a platelet rich plasma (PRP) method. In some embodiments, the platelet product has been subjected to photochemical treatment with a psoralen compound after preparation of the platelet product. In some embodiments, the platelet product has been subjected to photochemical treatment with a psoralen compound during preparation of the platelet product (e.g., treatment of platelet component(s)). In some embodiments, the one or more platelet components of the platelet product have been subjected to photochemical treatment with a psoralen compound.

In some embodiments of the aforementioned methods, the present disclosure sets forth a method of providing a platelet product to a subject in need thereof, comprising administering to a human subject with elevated risk for a leukocyte-related transfusion complication a platelet product, wherein the platelet product comprises one or more platelet components (e.g., whole blood derived platelet components, platelet concentrates, platelet preparations) and the one or more platelet components are prepared (e.g., from whole blood donation(s)) by a buffy coat method or a platelet rich plasma (PRP) method, and wherein the platelet product has been subjected to photochemical treatment with a psoralen compound (e.g., psoralen derivative, salt thereof) to inactivate pathogens and leukocytes, if present, and wherein the platelet product has not been subjected to treatment with either ionizing radiation (e.g., gamma irradiation, X-ray irradiation, X-irradiation) or a filtration medium (e.g., during preparation of the platelet product, after preparation of the platelet product) to reduce the level of contaminating leukocytes. In some embodiments, the one or more platelet components are prepared by a buffy coat method. In some embodiments, the one or more platelet components are prepared by a PRP method. In some embodiments, the platelet product has been subjected to photochemical treatment with a psoralen compound. In some embodiments, the platelet product has been subjected to photochemical treatment with a psoralen compound during preparation of the platelet product. In some embodiments, the one or more platelet components of the platelet product have been subjected to photochemical treatment with a psoralen compound.

In some embodiments of the aforementioned methods, the present disclosure sets forth a method of providing a platelet product to a subject in need thereof, comprising administering to a human subject with elevated risk for a leukocyte-related transfusion complication a platelet product, wherein the platelet product comprises one or more whole blood-derived platelet components prepared from whole blood donation(s); wherein the platelet product or platelet component(s) therein have been subjected to photochemical treatment with a psoralen compound to inactivate pathogens and leukocytes, if present; and wherein the platelet product or platelet component(s) therein have not been subjected to treatment with either ionizing radiation or a filtration medium to reduce the level of contaminating leukocytes. In some embodiments, the one or more platelet components are prepared by a buffy coat method. In some embodiments, the one or more platelet components are prepared by a PRP method.

In some embodiments of the aforementioned methods, the psoralen compound is a 4′-primary amino-substituted psoralen or 5′-primary amino-substituted psoralen. In some embodiments, the psoralen compound is a compound of formula I:

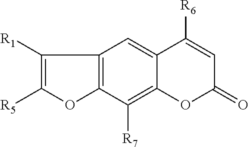

wherein in formula I, R₁ is
(CH₂)₂—NH₂,
—(CH₂)_{w}—R₂—(CH₂)_{z}—NH₂,
—(CH₂)_{w}—R₂—(CH₂)ₓ—R₃—(CH₂)_{z}—NH₂, or
(CH₂)_{w}—R₂—(CH₂)ₓ—R₃—(CH₂)_{y}—R₄—(CH₂)_{z}—NH₂;
   and R₂, R₃, and R₄ are independently O or NH, and w is a whole number from 1 to 5, x is a whole number from 2 to 5, y is a whole number from 2 to 5, and z is a whole number from 2 to 6; R₅, R₆ and R₇ are independently H or (CH₂)ᵥCH₃, and v is a whole number from 0 to 5. In some embodiments, the psoralen compound is 4′-(4-amino-2-aza)butyl-4,5′,8-trimethylpsoralen, 4′-(4-amino-2-oxa)butyl-4,5′,8-trimethylpsoralen, 4′-(2-aminoethyl)-4,5′,8-trimethylpsoralen, 4′-(5-amino-2-oxa)pentyl-4,5′,8-trimethylpsoralen, 4′-(5-amino-2-aza)pentyl-4, 5′8-trimethylpsoralen, 4′-(6-amino-2-aza)hexyl-4,5′,8-trimethylpsoralen, 4′-(7-amino-2,5-oxa)heptyl-4,5′,8-trimethylpsoralen, 4′-(12-amino-8-aza-2,5-dioxa)dodecyl-4,5′,8-trimethylpsoralen, 4′-(13-amino-2-aza-6,11-dioxa)tridecyl-4,5′,8-trimethylpsoralen, 4′-(7-amino-2-aza)heptyl-4,5′,8-trimethylpsoralen, 4′-(7-amino-2-aza-5-oxa)heptyl-4,5′,8-trimethylpsoralen, 4′-(9-amino-2,6-diaza)nonyl-4,5′,8-trimethylpsoralen, 4′-(8-amino-5-aza-2-oxa)octyl-4,5′,8-trimethylpsoralen, 4′-(9-amino-5-aza-2-oxa)nonyl-4,5′,8-trimethylpsoralen, or 4′-(14-amino-2,6,11-triaza)tetradecyl-4,5′,8-trimethylpsoralen. In some embodiments, the psoralen compound is 4′-(4-amino-2-oxa)butyl-4,5′,8-trimethylpsoralen, which may also be referred to as 3-(2-Aminoethoxymethyl)-2,5,9-trimethyl-7H-furo[3,2-g]-1-benzopyran-7-one hydrochloride, or S-59, or amotosalen.

In some embodiments of the aforementioned methods, the platelet product is a pooled platelet product, comprising a mixture of at least two platelet components. In some embodiments, the pooled platelet product comprises a mixture of 2 to 10 platelet components. In some embodiments, the pooled platelet product comprises a mixture of 3 to 10 platelet components. In some embodiments, the pooled platelet product comprises a mixture of 4 to 10 platelet components. In some embodiments, the pooled platelet product comprises a mixture of 4 to 9 platelet components. In some embodiments, the pooled platelet product comprises a mixture of 4 to 8 platelet components. In some embodiments, the pooled platelet product comprises a mixture of 4 to 7 platelet components. In some embodiments, the pooled platelet product comprises a mixture of 4 to 6 platelet components. In some embodiments, the pooled platelet product comprises a mixture of 3 platelet components, a mixture of 4 platelet components, a mixture of 5 platelet components, a mixture of 6 platelet components, a mixture of 7 platelet components or a mixture of 8 platelet components. In some embodiments the platelet components in the pooled platelet product are ABO blood group matched. In some embodiments the platelet components in the pooled platelet product are HLA matched.

In some embodiments of the aforementioned methods, the one or more platelet components each comprises about 0.1×10¹¹ to about 2.2×10¹¹ platelets. In some embodiments, the one or more platelet components each comprises about 0.4×10¹¹ to about 1.1×10¹¹ platelets. In some embodiments, the one or more platelet components each comprises about 0.4×10¹¹ to about 0.8×10¹¹ platelets. In some embodiments, the one or more platelet components each comprises about 0.6×10¹¹ to about 0.8×10¹¹ platelets. In some embodiments, the one or more platelet components each comprises about 0.8×10¹¹ to about 1.1×10¹¹ platelets. In some embodiments, the one or more platelet components each comprises about 0.8×10¹¹ platelets. In some embodiments, the one or more platelet components each comprises about 0.9×10¹¹ platelets. In some embodiments, the one or more platelet components each comprises about 1.0×10¹¹ platelets. In some embodiments, the one or more platelet components each comprises about 1.1×10¹¹ platelets.

In some embodiments of the aforementioned methods, the platelet product (e.g., pooled platelet product) comprises about 1.0×10¹¹ to about 8×10¹¹ platelets. In some embodiments, the platelet product comprises about 2.0×10¹¹ to about 8.0×10¹¹ platelets. In some embodiments, the platelet product comprises about 2.0×10¹¹ to about 7.0×10¹¹ platelets. In some embodiments, the platelet product comprises about 2.5×10¹¹ to about 7.0×10¹¹ platelets. In some embodiments, the platelet product comprises about 2.5×10¹¹ to about 6.0×10¹¹ platelets. In some embodiments, the platelet product comprises about 2.5×10¹¹ to about 5.0×10¹¹ platelets. In some embodiments, the platelet product comprises about 2.5×10¹¹ to about 4.0×10¹¹ platelets. In some embodiments, the platelet product comprises about 3.0×10¹¹ to about 7.0×10¹¹ platelets. In some embodiments, the platelet product comprises about 3.0×10¹¹ to about 6.0×10¹¹ platelets. In some embodiments, the platelet product comprises about 3.0×10¹¹ to about 5.0×10¹¹ platelets. In some embodiments, the platelet product comprises about 3.0×10¹¹ to about 4.0×10¹¹ platelets. In some embodiments, the platelet product comprises about 4.0×10¹¹ to about 6.4×10¹¹ platelets. In some embodiments, the platelet product comprises about 4.0×10¹¹ to about 7.0×10¹¹ platelets. In some embodiments, the platelet product comprises about 4.0×10¹¹ to about 6.0×10¹¹ platelets. In some embodiments, the platelet product comprises about 6.0×10¹¹ to about 7.0×10¹¹ platelets.

In some embodiments of the aforementioned methods, the platelet product further comprises donor plasma. In some embodiments, the platelet product comprises platelets in 100% plasma. In some embodiments of the aforementioned methods, the platelet product further comprises an additive solution. In some embodiments of the aforementioned methods, the platelet product comprises about 5 to 50% plasma and about 95 to 50% additive solution. In some embodiments, the platelet product comprises about 30-50, plasma and about 50-70% additive solution. In some embodiments, the platelet product comprises about 30%, about 35%, about 40, about 45% or about 50% plasma and the remainder as additive solution. In some embodiments, the platelet product comprises greater than 95% additive solution. In some embodiments, the additive solution is a platelet additive solution. In some embodiments of the aforementioned methods, the platelet product has not been subjected to treatment with a leukocyte reduction filter. In some embodiments of the aforementioned methods, the platelet product is a transfusion ready platelet product. In some embodiments, the transfusion ready platelet product comprises a platelet product in a storage container with suitable labeling for human use, wherein the labeling indicates that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the transfusion ready platelet product comprises a platelet product in a storage container and instructions for human use, wherein the instructions indicate that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the labeling and/or instructions indicate that the transfusion ready platelet product has not been subjected to treatment with either ionizing radiation (e.g., gamma irradiation, X-ray irradiation, X-irradiation) or a filtration medium to reduce the level of contaminating leukocytes. In some embodiments of the aforementioned methods, the subject with elevated risk for a leukocyte-related transfusion complication is a recipient of peripheral blood stem cell or bone marrow transplant. In some embodiments of the aforementioned methods, the subject with elevated risk for a leukocyte-related transfusion complication is a recipient of ablative chemotherapy and/or radiotherapy. In some embodiments of the aforementioned methods, the subject with elevated risk for a leukocyte-related transfusion complication is a subject diagnosed with a hematologic malignancy or solid tumor. In some embodiments of the aforementioned methods, the subject with elevated risk for a leukocyte-related transfusion complication is a subject diagnosed with an immunodeficiency. In some embodiments, the immunodeficiency is a congenital immunodeficiency.

In some embodiments of the aforementioned methods, the method comprises administering the platelet product (e.g., an aforementioned platelet product, a platelet product of any of the preceding embodiments) to the subject 2 or more times. In some embodiments, the method comprises administering an aforementioned platelet product to the subject 3 or more times, 4 or more times or 5 or more times. In some embodiments, the method comprises administering to the subject 2 or more times, 3 or more times, 4 or more times or 5 or more times a platelet product of any of the preceding embodiments. In some embodiments of the aforementioned methods, the method comprises administering two or more transfusions (e.g., units, doses) (e.g., infusions) of the platelet product to the subject. In some embodiments, the method comprises administering three or more transfusions, 4 or more transfusions or 5 or more transfusions of the platelet product to the subject. In some embodiments, the method comprises administering to the subject two or more transfusions, three or more transfusions, four or more transfusions or five or more transfusions of a platelet product of any of the preceding embodiments.

In some embodiments of the aforementioned methods, the subject has a platelet count of less than about 20,000/μL prior to administration of the platelet product. In some embodiments, the subject has a platelet count of less than about 10,000/μL prior to administration of the platelet product. In some embodiments, the subject has a platelet count of less than about 5,000/μL prior to administration of the platelet product. In some embodiments of the aforementioned methods, administering the platelet product to the subject results in a 1 hour corrected count increment (CCI) of greater than 5,000. In some embodiments, administering the platelet product to the subject results in a 1 hour corrected count increment (CCI) of greater than 10,000. In some embodiments, administering the platelet product to the subject results in a 1 hour corrected count increment (CCI) of greater than 15,000. In some embodiments, administering the platelet product to the subject results in a 1 hour corrected count increment (CCI) of greater than 20,000 or more. In some embodiments of the aforementioned methods, the method reduces a leukocyte-related transfusion complication (e.g., after administration of the platelet product to a subject). In some embodiments, the leukocyte-related transfusion complication is transfusion-associated graft-versus-host disease (TA-GVHD). In some embodiments, the leukocyte-related transfusion complication is alloimmunization. In some embodiments, the leukocyte-related transfusion complication is alloimmune platelet refractoriness. In some embodiments, the leukocyte-related transfusion complication is cytokine stimulation and/or production. In some embodiments, the leukocyte-related transfusion complication is microchimerism. In some embodiments, the leukocyte-related transfusion complication is a febrile non-hemolytic transfusion reaction (FNHTR).

In another aspect, the present disclosure provides a method of preparing a transfusion ready platelet product for administration to a subject (e.g., subject with elevated risk for a leukocyte-related transfusion complication) in need thereof, comprising: a) preparing from whole blood donation(s) one or more platelet components (e.g., whole blood-derived platelet components, isolated platelet components, platelet concentrates, platelet preparations); b) subjecting the one or more platelet components to photochemical treatment with a psoralen compound to inactivate pathogens and leukocytes, if present, comprising adding to the one or more platelet components the psoralen compound and exposing the mixture of platelet component(s) and psoralen compound to ultraviolet A light; and c) transferring the photochemical treated platelet component(s) to one or more storage containers with suitable labeling for human use, to provide a transfusion ready platelet product. In some embodiments, the method further comprises, d) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration, leukocyte reduction filter).

In another aspect, the present disclosure provides a method of preparing a transfusion ready platelet product for administration to a subject (e.g., subject with elevated risk for a leukocyte-related transfusion complication) in need thereof, comprising:
a) preparing from whole blood donation(s) one or more platelet components (e.g., whole blood-derived platelet components, isolated platelet components, platelet concentrates, platelet preparations);
b) subjecting the one or more platelet components to photochemical treatment with a psoralen compound to inactivate pathogens and leukocytes, if present, comprising adding to the one or more platelet components the psoralen compound and exposing the mixture of platelet component(s) and psoralen compound to ultraviolet A light;
c) transferring the photochemical treated platelet component(s) to one or more storage containers with suitable labeling for human use, to provide a transfusion ready platelet product; and
d) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration).

In some embodiments of the aforementioned methods, the one or more platelet components are prepared by a buffy coat method. In some of the aforementioned embodiments, the one or more platelet components are prepared by a platelet rich plasma (PRP) method. In some embodiments, the whole blood donations have not been subjected to treatment with ionizing radiation (e.g., gamma irradiation, X-ray irradiation, X-irradiation) or a filtration media (e.g., leukofiltration) (e.g., to reduce the level of contaminating leukocytes). In some embodiments, the platelet components have not been subjected to treatment with ionizing radiation (e.g., gamma irradiation, X-ray irradiation, X-irradiation) or a filtration media (e.g., leukofiltration) (e.g., to reduce the level of contaminating leukocytes).

In some embodiments of the aforementioned methods, the one or more storage containers with suitable labeling for human use comprises labeling indicating that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the one or more storage containers with suitable labeling for human use includes instructions indicating that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the labeling and/or instructions indicate that the transfusion ready platelet product has not been subjected to treatment with either ionizing radiation (e.g., gamma irradiation, X-ray irradiation, X-irradiation) or a filtration medium to reduce the level of contaminating leukocytes.

In another aspect, the present disclosure provides a method of preparing a transfusion ready platelet product for administration to a subject in need thereof, comprising: a) preparing from whole blood donations a plurality of platelet components (e.g., whole blood-derived platelet components, isolated platelet components, platelet concentrates, platelet preparations); b) pooling two or more of the platelet components to produce a pooled platelet product; c) subjecting the pooled platelet product or each platelet component (e.g., prior to pooling) to photochemical treatment with a psoralen compound to inactivate pathogens and leukocytes, if present, comprising adding to the pooled platelet product or each platelet component the psoralen compound and exposing the mixture of pooled platelet product or platelet component and psoralen compound to ultraviolet A light; and d) transferring the photochemical treated pooled platelet product to a storage container (e.g., with suitable labeling for human use) to provide a transfusion ready platelet product (e.g., for administration to a human subject in need thereof). In some embodiments, the method further comprises, e) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration, leukocyte reduction filter) to reduce the level of contaminating leukocytes.

In another aspect, the present disclosure provides a method of preparing a transfusion ready platelet product for administration to a subject in need thereof, comprising (e.g., comprising the steps of):
a) preparing from whole blood donations a plurality of platelet components (e.g., whole blood-derived platelet components, isolated platelet components, platelet concentrates, platelet preparations),
b) pooling two or more of the platelet components to produce a pooled platelet product;
c) subjecting the pooled platelet product or each platelet component (e.g., prior to pooling) to photochemical treatment with a psoralen compound to inactivate pathogens and leukocytes, if present, comprising adding to the pooled platelet product or each platelet component the psoralen compound and exposing the mixture of pooled platelet product or platelet component and psoralen compound to ultraviolet A light;
d) transferring the photochemical treated pooled platelet product to a storage container (e.g., with suitable labeling for human use) to provide a transfusion ready platelet product (e.g., for administration to a human subject in need thereof); and e) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration) to reduce the level of contaminating leukocytes.

In some embodiments of the aforementioned methods, the plurality of platelet components is prepared by a buffy coat method. In some of the aforementioned embodiments, the plurality of platelet components is prepared by a platelet rich plasma (PRP) method. In some embodiments, the platelet components are subjected to photochemical treatment with a psoralen compound prior to pooling. In some embodiments, the pooled platelet product is subjected to photochemical treatment with a psoralen compound. In some embodiments, the whole blood donations have not been subjected to treatment with ionizing radiation (e.g., gamma irradiation, X-ray irradiation, X-irradiation) or a filtration media (e.g., leukofiltration) (e.g., to reduce the level of contaminating leukocytes). In some embodiments, the platelet components have not been subjected to treatment with ionizing radiation (e.g., gamma irradiation, X-ray irradiation, X-irradiation) or a filtration media (e.g., leukofiltration) (e.g., to reduce the level of contaminating leukocytes).

In some embodiments of the aforementioned methods, the storage container (e.g., storage container with suitable labeling for human use) comprises labeling indicating that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the storage container with suitable labeling for human use includes instructions indicating that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the labeling and/or instructions indicate that the transfusion ready platelet product has not been subjected to treatment with either ionizing radiation (e.g., gamma irradiation, X-ray irradiation, X-irradiation) or a filtration medium to reduce the level of contaminating leukocytes.

In some embodiments of the aforementioned methods, the present disclosure provides a method of preparing a transfusion ready platelet product for administration to a subject in need thereof, comprising (e.g., comprising the steps of):
a) preparing from whole blood donations a plurality of platelet components (e.g., whole blood-derived platelet components, isolated platelet components, platelet concentrates, platelet preparations) by a buffy coat or platelet rich plasma (PRP) method;
b) pooling two or more of the platelet components prepared by the buffy coat method or two or more of the platelet components prepared by the PRP method, to produce a pooled platelet product;
c) subjecting the pooled platelet product or each platelet component to photochemical treatment with a psoralen compound to inactivate pathogens and leukocytes, if present, comprising adding to the pooled platelet product or each platelet component the psoralen compound and exposing the mixture of pooled platelet product or platelet component and psoralen compound to ultraviolet A light;
d) transferring the photochemical treated pooled platelet product to a storage container to provide a transfusion ready platelet product (e.g., for administration to a human subject in need thereof); and
e) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration) to reduce the level of contaminating leukocytes.

In some embodiments of the aforementioned methods, the method of preparing a transfusion ready platelet product for administration to a subject in need thereof comprises:
a) preparing from whole blood donations a plurality of platelet components (e.g., whole blood-derived platelet components) by a buffy coat or PRP method;
b) subjecting each platelet component to photochemical treatment with a psoralen compound to inactivate pathogens and leukocytes, if present, comprising adding to each platelet component the psoralen compound and exposing the mixture of platelet component and psoralen compound to ultraviolet A light;
c) pooling two or more of the platelet components prepared by the buffy coat method or two or more of the platelet components prepared by the PRP method, to produce a pooled platelet product;
d) transferring the photochemical treated pooled platelet product to a storage container to provide a transfusion ready platelet product; and
e) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration) to reduce the level of contaminating leukocytes.

In some embodiments, the transfusion ready platelet product is suitable for administration to a human subject with elevated risk for a leukocyte-related transfusion complication. In some embodiments, the method is a method of preparing a transfusion ready platelet product for administration to a subject in need thereof, comprising the steps of:
a) preparing from whole blood donations a plurality of platelet components (e.g., whole blood-derived platelet components) by a buffy coat or PRP method;
b) subjecting each platelet component to photochemical treatment with a psoralen compound to inactivate pathogens and leukocytes, if present, comprising adding to each platelet component the psoralen compound and exposing the mixture of platelet component and psoralen compound to ultraviolet A light;
c) pooling two or more of the platelet components prepared by the buffy coat method or two or more of the platelet components prepared by the PRP method, to produce a pooled platelet product;
d) transferring the photochemical treated pooled platelet product to a storage container to provide a transfusion ready platelet product; and
e) providing the transfusion ready platelet product for administration to a human subject in need thereof with elevated risk for a leukocyte-related transfusion complication, without a further step of treatment with either ionizing radiation (e.g., gamma irradiation, X-ray irradiation, X-irradiation) or a filtration medium (e.g., leukofiltration) to reduce the level of contaminating leukocytes.

In some embodiments of the aforementioned methods, the method of preparing a transfusion ready platelet product for administration to a subject in need thereof comprises:
a) preparing from whole blood donations a plurality of platelet components (e.g., whole blood-derived platelet components) by a buffy coat or PRP method;
b) pooling two or more of the platelet components prepared by the buffy coat method or two or more of the platelet components prepared by the PRP method, to produce a pooled platelet product;
c) subjecting the pooled platelet product to photochemical treatment with a psoralen compound to inactivate pathogens and leukocytes, if present, comprising adding to the pooled platelet product the psoralen compound and exposing the mixture of pooled platelet product and psoralen compound to ultraviolet A light;
d) transferring the photochemical treated pooled platelet product to a storage container to provide a transfusion ready platelet product; and
e) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration) to reduce the level of contaminating leukocytes.

In some embodiments, the transfusion ready platelet product is suitable for administration to a human subject with elevated risk for a leukocyte-related transfusion complication. In some embodiments, the method is a method of preparing a transfusion ready platelet product for administration to a subject in need thereof, comprising the steps of:
a) preparing from whole blood donations a plurality of platelet components (e.g., whole blood-derived platelet components) by a buffy coat or PRP method;
b) pooling two or more of the platelet components prepared by the buffy coat method or two or more of the platelet components prepared by the PRP method, to produce a pooled platelet product;
c) subjecting the pooled platelet product to photochemical treatment with a psoralen compound to inactivate pathogens and leukocytes, if present, comprising adding to the pooled platelet product the psoralen compound and exposing the mixture of pooled platelet product and psoralen compound to ultraviolet A light;
d) transferring the photochemical treated pooled platelet product to a storage container to provide a transfusion ready platelet product; and
e) providing the transfusion ready platelet product for administration to a human subject in need thereof with elevated risk for a leukocyte-related transfusion complication, without a further step of treatment with either ionizing radiation (e.g., gamma irradiation, X-ray irradiation, X-irradiation) or a filtration medium (e.g., leukofiltration) to reduce the level of contaminating leukocytes.

In some embodiments of the aforementioned methods, the transfusion ready platelet product reduces leukocyte-related transfusion complications (e.g., the risk of leukocyte-related transfusion complications) in a recipient of a platelet product (e.g., the transfusion ready platelet product). In some embodiments of the aforementioned methods, the subject with elevated risk for a leukocyte-related transfusion complication is a recipient of a peripheral blood stem cell or bone marrow transplant. In some embodiments, the transfusion ready platelet product is suitable for administration to a human subject that is a recipient of a peripheral blood stem cell or bone marrow transplant. In some embodiments, the subject with elevated risk for a leukocyte-related transfusion complication is a recipient of ablative chemotherapy and/or radiotherapy. In some embodiments, the transfusion ready platelet product is suitable for administration to a human subject that is a recipient of ablative chemotherapy and/or radiotherapy. In some embodiments, the subject with elevated risk for a leukocyte-related transfusion complication is a subject diagnosed with a hematologic malignancy or solid tumor. In some embodiments, the transfusion ready platelet product is suitable for administration to a human subject that is diagnosed with a hematologic malignancy or solid tumor. In some embodiments, the subject with elevated risk for a leukocyte-related transfusion complication is a subject diagnosed with an immunodeficiency. In some embodiments, the transfusion ready platelet product is suitable for administration to a human subject that is a subject diagnosed with an immunodeficiency. In some embodiments, the immunodeficiency is a congenital immunodeficiency.

In some embodiments of the aforementioned methods, the platelet component(s) (e.g., one or more platelet components, plurality of platelet components) is prepared by a buffy coat method. In some of the aforementioned embodiments, the platelet component(s) (e.g., one or more platelet components, plurality of platelet components) is prepared by a platelet rich plasma (PRP) method. In some embodiments, the platelet components are subjected to photochemical treatment with a psoralen compound prior to pooling. In some embodiments, the pooled platelet product is subjected to photochemical treatment with a psoralen compound. In some embodiments, the transfusion ready platelet product is subjected to photochemical treatment with a psoralen compound. In some embodiments, the whole blood donations have not been subjected to treatment with ionizing radiation (e.g., gamma irradiation, X-ray irradiation, X-irradiation) or a filtration media (e.g., leukofiltration) (e.g., to reduce the level of contaminating leukocytes). In some embodiments, the platelet components have not been subjected to treatment with ionizing radiation (e.g., gamma irradiation, X-ray irradiation, X-irradiation) or a filtration media (e.g., leukofiltration) (e.g., to reduce the level of contaminating leukocytes). In some embodiments, the pooled platelet product has not been subjected to treatment with ionizing radiation (e.g., gamma irradiation, X-ray irradiation, X-irradiation) or a filtration media (e.g., leukofiltration) (e.g., to reduce the level of contaminating leukocytes). In some embodiments, the transfusion ready platelet product has not been subjected to treatment with ionizing radiation (e.g., gamma irradiation, X-ray irradiation, X-irradiation) or a filtration media (e.g., leukofiltration) (e.g., to reduce the level of contaminating leukocytes). In some embodiments, the storage container comprises labeling indicating that treatment with ionizing radiation (e.g., gamma irradiation, X-ray irradiation, X-irradiation) or a filtration media (e.g., leukocyte reduction filter) is not required prior to administration of the platelet product to a subject (e.g., human subject). In some embodiments, the transfusion ready platelet product or storage container further comprising instructions indicating that treatment with ionizing radiation or a leukocyte reduction filter is not required prior to administration of the platelet product to a subject (e.g., human subject). In some embodiments, the labeling and/or the instructions indicate that the transfusion ready platelet product has not been subjected to treatment with either ionizing radiation or a leukocyte reduction filter. In some embodiments, the method further comprises e) providing the transfusion ready platelet product for administration to a human subject in need thereof with elevated risk for a leukocyte-related transfusion complication, without a further step of treatment with either gamma irradiation or a filtration medium to reduce the level of contaminating leukocytes. In some embodiments, the transfusion ready platelet product is administered (e.g., infused, transfused) to a subject (e.g, subject in need thereof).

In some embodiments of the aforementioned methods, the psoralen compound is a 4′-primary amino-substituted psoralen or 5′-primary amino-substituted psoralen. In some embodiments, the psoralen compound is a compound of formula I:

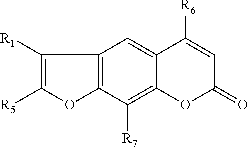

wherein in formula I, R₁ is
(CH₂)₂—NH₂,
—(CH₂)_{w}—R₂—(CH₂)_{z}—NH₂,
—(CH₂)_{w}—R₂—(CH₂)ₓ—R₃—(CH₂)_{z}—NH₂, or
(CH₂)_{w}—R₂—(CH₂)ₓ—R₃—(CH₂)_{y}—R₄—(CH₂)_{z}—NH₂;
   and R₂, R₃, and R₄ are independently O or NH, and w is a whole number from 1 to 5, x is a whole number from 2 to 5, y is a whole number from 2 to 5, and z is a whole number from 2 to 6; R₅, R₆ and R₇ are independently H or (CH₂)ᵥCH₃, and v is a whole number from 0 to 5. In some embodiments, the psoralen compound is 4′-(4-amino-2-aza)butyl-4,5′,8-trimethylpsoralen, 4′-(4-amino-2-oxa)butyl-4,5′,8-trimethylpsoralen, 4′-(2-aminoethyl)-4,5′,8-trimethylpsoralen, 4′-(5-amino-2-oxa)pentyl-4,5′,8-trimethylpsoralen, 4′-(5-amino-2-aza)pentyl-4,5′8-trimethylpsoralen, 4′-(6-amino-2-aza)hexyl-4,5′,8-trimethylpsoralen, 4′-(7-amino-2,5-oxa)heptyl-4,5′,8-trimethylpsoralen, 4′-(12-amino-8-aza-2,5-dioxa)dodecyl-4,5′,8-trimethylpsoralen, 4′-(13-amino-2-aza-6,11-dioxa)tridecyl-4,5′,8-trimethylpsoralen, 4′-(7-amino-2-aza)heptyl-4,5′,8-trimethylpsoralen, 4′-(7-amino-2-aza-5-oxa)heptyl-4,5′,8-trimethylpsoralen, 4′-(9-amino-2,6-diaza)nonyl-4,5′,8-trimethylpsoralen, 4′-(8-amino-5-aza-2-oxa)octyl-4,5′,8-trimethylpsoralen, 4′-(9-amino-5-aza-2-oxa)nonyl-4,5′,8-trimethylpsoralen, or 4′-(14-amino-2,6,11-triaza)tetradecyl-4,5′,8-trimethylpsoralen. In some embodiments, the psoralen compound is 4′-(4-amino-2-oxa)butyl-4,5′,8-trimethylpsoralen, which may also be referred to as 3-(2-Aminoethoxymethyl)-2,5,9-trimethyl-7H-furo[3,2-g]-1-benzopyran-7-one hydrochloride, or S-59, or amotosalen.

In some embodiments of the aforementioned methods, the method comprises pooling at least 3, at least 4, at least 5, at least 6, at least 7 or at least 8 platelet components. In some embodiments, the method comprises pooling 2 to 10 platelet components. In some embodiments, the method comprises pooling 3 to 10 platelet components. In some embodiments, the method comprises pooling 4 to 10 platelet components. In some embodiments, the method comprises pooling 4 to 9 platelet components. In some embodiments, the method comprises pooling 4 to 8 platelet components. In some embodiments, the method comprises pooling 4 to 7 platelet components. In some embodiments, the method comprises pooling 4 to 6 platelet components. In some embodiments, the method comprises pooling 3 platelet components, pooling 4 platelet components, pooling 5 platelet components, pooling 6 platelet components, pooling 7 platelet components or pooling 8 platelet components. In some embodiments the platelet components in the pooled platelet product are ABO blood group matched. In some embodiments the platelet components in the pooled platelet product are HLA matched.

In some embodiments of the aforementioned methods, the platelet components each comprise about 0.1×10¹¹ to about 2.2×10¹¹ platelets. In some embodiments, the platelet components each comprise about 0.4×10¹¹ to about 1.1×10¹¹ platelets. In some embodiments, the platelet components each comprise about 0.4×10¹¹ to about 0.8×10¹¹ platelets. In some embodiments, the platelet components each comprise about 0.6×10¹¹ to about 0.8×10¹¹ platelets. In some embodiments, the platelet components each comprise about 0.8×10¹¹ to about 1.1×10¹¹ platelets. In some embodiments, the platelet components each comprise about 0.8×10¹¹ platelets. In some embodiments, the platelet components each comprises about 0.9×10¹¹ platelets. In some embodiments, the platelet components each comprise about 1.0×10¹¹ platelets. In some embodiments, the platelet components each comprise about 1.1×10¹¹ platelets.

In some embodiments of the aforementioned methods, the transfusion ready platelet product comprises about 1.0×10¹¹ to about 8×10¹¹ platelets. In some embodiments, the transfusion ready platelet product comprises about 2.0×10¹¹ to about 8.0×10¹¹ platelets. In some embodiments, the transfusion ready platelet product comprises about 2.0×10¹¹ to about 7.0×10¹¹ platelets. In some embodiments, the transfusion ready platelet product comprises about 2.5×10¹¹ to about 7.0×10¹¹ platelets. In some embodiments, the transfusion ready platelet product comprises about 2.5×10¹¹ to about 6.0×10¹¹ platelets. In some embodiments, the transfusion ready platelet product comprises about 2.5×10¹¹ to about 5.0×10¹¹ platelets. In some embodiments, the transfusion ready platelet product comprises about 2.5×10¹¹ to about 4.0×10¹¹ platelets. In some embodiments, the transfusion ready platelet product comprises about 3.0×10¹¹ to about 7.0×10¹¹ platelets. In some embodiments, the transfusion ready platelet product comprises about 3.0×10¹¹ to about 6.0×10¹¹ platelets. In some embodiments, the transfusion ready platelet product comprises about 3.0×10¹¹ to about 5.0×10¹¹ platelets. In some embodiments, the transfusion ready platelet product comprises about 3.0×10¹¹ to about 4.0×10¹¹ platelets. In some embodiments, the transfusion ready platelet product comprises about 4.0×10¹¹ to about 6.4×10¹¹ platelets. In some embodiments, the transfusion ready platelet product comprises about 4.0×10¹¹ to about 7.0×10¹¹ platelets. In some embodiments, the transfusion ready platelet product comprises about 4.0×10¹¹ to about 6.0×10¹¹ platelets. In some embodiments, the transfusion ready platelet product comprises about 6.0×10¹¹ to about 7.0×10¹¹ platelets.

In some embodiments of the aforementioned methods, the transfusion ready platelet product further comprises donor plasma. In some embodiments, the platelet product comprises platelets in 100% plasma. In some embodiments of the aforementioned methods, the transfusion ready platelet product further comprises an additive solution. In some embodiments of the aforementioned methods, the transfusion ready platelet product comprises about 5 to 50% plasma and about 95 to 50% additive solution. In some embodiments, the transfusion ready platelet product comprises about 30-50% plasma and about 50-70% additive solution. In some embodiments, the transfusion ready platelet product comprises about 30/o, about 35%, about 40%, about 45% or about 50% plasma and the remainder as additive solution. In some embodiments, the transfusion ready platelet product comprises greater than 95% additive solution. In some embodiments, the additive solution is a platelet additive solution. In some embodiments of the aforementioned methods, the transfusion ready platelet product comprises a platelet product in a storage container with suitable labeling for human use, wherein the labeling indicates that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the transfusion ready platelet product comprises a platelet product in a storage container and instructions for human use, wherein the instructions indicate that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the labeling and/or instructions indicate that the transfusion ready platelet product has not been subjected to treatment with either ionizing radiation (e.g., gamma irradiation, X-ray irradiation, X-irradiation) or a filtration medium to reduce the level of contaminating leukocytes.

In another aspect, the present disclosure sets forth a method of providing a platelet product to a subject in need thereof, comprising administering to a human subject a transfusion ready platelet product prepared according to any of the aforementioned methods. In some embodiments, the subject is a human subject with elevated risk for a leukocyte-related transfusion complication. In some embodiments, the method comprises administering the transfusion ready platelet product to the subject 2 or more times. In some embodiments, the method comprises administering the transfusion ready platelet product to the subject 3 or more times, 4 or more times or 5 or more times. In some embodiments, the method comprises administering two or more transfusions (e.g., units, doses) of the transfusion ready platelet product to the subject. In some embodiments, the method comprises administering three or more transfusions, 4 or more transfusions or 5 or more transfusions of the transfusion ready platelet product to the subject.

In some embodiments of the aforementioned methods, the subject has a platelet count of less than about 20,000/μL prior to administration of the transfusion ready platelet product. In some embodiments, the subject has a platelet count of less than about 10,000/L prior to administration of the transfusion ready platelet product. In some embodiments, the subject has a platelet count of less than about 5,000/μL prior to administration of the transfusion ready platelet product.

In some embodiments of the aforementioned methods, administering the transfusion ready platelet product to the subject results in a 1 hour corrected count increment (CCI) of greater than 5,000. In some embodiments, administering the transfusion ready platelet product to the subject results in a 1 hour corrected count increment (CCI) of greater than 10,000. In some embodiments, administering the transfusion ready platelet product to the subject results in a 1 hour corrected count increment (CCI of greater than 15,000. In some embodiments, administering the transfusion ready platelet product to the subject results in a 1 hour corrected count increment (CCI) of greater than 20,000 or more.

In some embodiments of the aforementioned methods, the method reduces a leukocyte-related transfusion complication (e.g., after administration of the platelet product to a subject). In some embodiments, the leukocyte-related transfusion complication is transfusion-associated graft-versus-host disease (TA-GVHD). In some embodiments, the leukocyte-related transfusion complication is alloimmunization. In some embodiments, the leukocyte-related transfusion complication is alloimmune platelet refractoriness. In some embodiments, the leukocyte-related transfusion complication is cytokine stimulation and/or production. In some embodiments, the leukocyte-related transfusion complication is microchimerism. In some embodiments, the leukocyte-related transfusion complication is a febrile non-hemolytic transfusion reaction (FNHTR).

In another aspect, the present disclosure sets forth a method of treating a subject in need thereof with a platelet product, comprising administering to a human subject a transfusion ready platelet product prepared according to any of the aforementioned methods. In some embodiments, the subject is a subject with elevated risk for a leukocyte-related transfusion complication. In some embodiments, the method comprises administering the transfusion ready platelet product 2 or more times, 3 or more times, 4 or more times or 5 or more times.

In some embodiments of the aforementioned methods, the subject has a platelet count of less than about 20,000/μL prior to administration of the transfusion ready platelet product. In some embodiments, the subject has a platelet count of less than about 10,000/L prior to administration of the transfusion ready platelet product. In some embodiments, the subject has a platelet count of less than about 5,000/μL prior to administration of the transfusion ready platelet product.

In some embodiments of the aforementioned methods, administering the transfusion ready platelet product to the subject results in a 1 hour corrected count increment (CCI) of greater than 5,000. In some embodiments, administering the transfusion ready platelet product to the subject results in a 1 hour corrected count increment (CCI) of greater than 10,000. In some embodiments, administering the transfusion ready platelet product to the subject results in a 1 hour corrected count increment (CCD of greater than 15,000. In some embodiments, administering the transfusion ready platelet product to the subject results in a 1 hour corrected count increment (CCI) of greater than 20,000 or more.

In some embodiments of the aforementioned methods, the method reduces a leukocyte-related transfusion complication (e.g., after administration of the platelet product to a subject). In some embodiments, the leukocyte-related transfusion complication is transfusion-associated graft-versus-host disease (TA-GVHD). In some embodiments, the leukocyte-related transfusion complication is alloimmunization. In some embodiments, the leukocyte-related transfusion complication is alloimmune platelet refractoriness. In some embodiments, the leukocyte-related transfusion complication is cytokine stimulation and/or production. In some embodiments, the leukocyte-related transfusion complication is microchimerism. In some embodiments, the leukocyte-related transfusion complication is a febrile non-hemolytic transfusion reaction (FNHTR).

In another aspect, the present disclosure provides a method for reducing leukocyte-related transfusion complications in a recipient of a platelet product, comprising:
a) preparing from whole blood donations a plurality of platelet components (e.g., whole blood-derived platelet components);
b) pooling two or more platelet components to produce a pooled platelet concentrate;
c) subjecting the pooled platelet product or each platelet component (e.g., prior to pooling) to photochemical treatment with a psoralen compound to inactivate pathogens and leukocytes, if present, comprising adding to the pooled platelet product or each platelet component the psoralen compound and exposing the mixture of pooled platelet product and psoralen compound or platelet component and psoralen compound to ultraviolet light;
d) transferring the photochemical treated pooled platelet product to a storage container (e.g., with suitable labeling for human use) to provide a transfusion ready platelet product; and
e) administering the transfusion ready platelet product to a human subject in need thereof, without a further step of treatment with either ionizing radiation (e.g., gamma irradiation, X-ray irradiation, X-irradiation) or a filtration medium (e.g., leukofiltration, leukocyte reduction filter) to reduce the level of contaminating leukocytes

In some embodiments of the aforementioned methods, the plurality of platelet components is prepared by a buffy coat method. In some embodiments of the aforementioned methods, the plurality of platelet components is prepared by a platelet rich plasma (PRP) method. In some embodiments, the platelet components are subjected to photochemical treatment with a psoralen compound prior to pooling. In some embodiments, the pooled platelet product is subjected to photochemical treatment with a psoralen compound. In some embodiments, the whole blood donations have not been subjected to treatment with ionizing radiation (e.g., gamma irradiation, X-ray irradiation, X-irradiation) or a filtration media (e.g., leukofiltration) (e.g., to reduce the level of contaminating leukocytes). In some embodiments, the platelet components have not been subjected to treatment with ionizing radiation (e.g., gamma irradiation, X-ray irradiation, X-irradiation) or a filtration media (e.g., leukofiltration) (e.g., to reduce the level of contaminating leukocytes).

In some embodiments of the aforementioned methods, the storage container (e.g., storage container with suitable labeling for human use) comprises labeling indicating that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the storage container with suitable labeling for human use includes instructions indicating that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the labeling and/or instructions indicate that the transfusion ready platelet product has not been subjected to treatment with either ionizing radiation (e.g., gamma irradiation, X-ray irradiation, X-irradiation) or a filtration medium to reduce the level of contaminating leukocytes.

In some embodiments of the aforementioned methods, the present disclosure provides a method for reducing leukocyte-related transfusion complications in a recipient of a platelet product, comprising:
a) preparing from whole blood donations a plurality of platelet components (e.g., whole blood-derived platelet components) by a buffy coat or PRP method;
b) pooling two or more platelet components prepared by the buffy coat method or two or more platelet components prepared by the PRP method to produce a pooled platelet concentrate;
c) subjecting the pooled platelet product or each platelet component to photochemical treatment with a psoralen compound to inactivate pathogens and leukocytes, if present, comprising adding to the pooled platelet product or each platelet component the psoralen compound and exposing the mixture of pooled platelet product or platelet component and psoralen compound to ultraviolet light;
d) transferring the photochemical treated pooled platelet product to a storage container to provide a transfusion ready platelet product; and
e) administering the transfusion ready platelet product to a human subject in need thereof, without a further step of treatment with either ionizing radiation (e.g., gamma irradiation, X-ray irradiation, X-irradiation) or a filtration medium (e.g., leukofiltration) to reduce the level of contaminating leukocytes.

In some embodiments, the method comprises:
a) preparing from whole blood donations a plurality of platelet components by a buffy coat or PRP method;
b) subjecting each platelet component to photochemical treatment with a psoralen compound to inactivate pathogens and leukocytes, if present, comprising adding to each platelet component the psoralen compound and exposing the mixture of platelet component and psoralen compound to ultraviolet A light;
c) pooling two or more of the platelet components prepared by the buffy coat method or two or more of the platelet components prepared by the PRP method, to produce a pooled platelet product;
d) transferring the photochemical treated pooled platelet product to a storage container to provide a transfusion ready platelet product; and
e) administering the transfusion ready platelet product to a human subject in need thereof, without a further step of treatment with either ionizing radiation (e.g., gamma irradiation, X-ray irradiation, X-irradiation) or a filtration medium (e.g., leukofiltration) to reduce the level of contaminating leukocytes.

In some embodiments, the method comprises:
a) preparing from whole blood donations a plurality of platelet components (e.g., whole blood-derived platelet components) by a buffy coat or PRP method;
b) pooling two or more of the platelet components prepared by the buffy coat method or two or more of the platelet components prepared by the PRP method, to produce a pooled platelet product;
c) subjecting the pooled platelet product to photochemical treatment with a psoralen compound to inactivate pathogens and leukocytes, if present, comprising adding to the pooled platelet product the psoralen compound and exposing the mixture of pooled platelet product and psoralen compound to ultraviolet A light;
d) transferring the photochemical treated pooled platelet product to a storage container to provide a transfusion ready platelet product; and
e) administering the transfusion ready platelet product to a human subject in need thereof, without a further step of treatment with either ionizing radiation (e.g., gamma irradiation, X-ray irradiation, X-irradiation) or a filtration medium (e.g., leukofiltration) to reduce the level of contaminating leukocytes.

In some embodiments of the aforementioned methods, the transfusion ready platelet product is suitable for administration to a human subject with elevated risk for a leukocyte-related transfusion complication. In some embodiments of the aforementioned methods, the recipient is a human subject with elevated risk for a leukocyte-related transfusion complication. In some embodiments, the subject with elevated risk for a leukocyte-related transfusion complication is a recipient of a peripheral blood stem cell or bone marrow transplant. In some embodiments, the transfusion ready platelet product is suitable for administration to a human subject that is a recipient of a peripheral blood stem cell or bone marrow transplant. In some embodiments, the subject with elevated risk for a leukocyte-related transfusion complication is a recipient of ablative chemotherapy and/or radiotherapy. In some embodiments, the transfusion ready platelet product is suitable for administration to a human subject that is a recipient of ablative chemotherapy and/or radiotherapy. In some embodiments, the subject with elevated risk for a leukocyte-related transfusion complication is a subject diagnosed with a hematologic malignancy or solid tumor. In some embodiments, the transfusion ready platelet product is suitable for administration to a human subject that is diagnosed with a hematologic malignancy or solid tumor. In some embodiments, the subject with elevated risk for a leukocyte-related transfusion complication is a subject diagnosed with an immunodeficiency. In some embodiments, the transfusion ready platelet product is suitable for administration to a human subject that is a subject diagnosed with an immunodeficiency. In some embodiments, the immunodeficiency is a congenital immunodeficiency.

In some embodiments of the aforementioned methods, the plurality of platelet components is prepared by a buffy coat method. In some embodiments of the aforementioned methods, the plurality of platelet components is prepared by a platelet rich plasma (PRP) method. In some embodiments, the platelet components are subjected to photochemical treatment with a psoralen compound prior to pooling. In some embodiments, the pooled platelet product is subjected to photochemical treatment with a psoralen compound. In some embodiments, the whole blood donations have not been subjected to treatment with ionizing radiation (e.g., gamma irradiation, X-ray irradiation, X-irradiation) or a filtration media (e.g., leukofiltration) (e.g., to reduce the level of contaminating leukocytes). In some embodiments, the platelet components have not been subjected to treatment with ionizing radiation (e.g., gamma irradiation, X-ray irradiation, X-irradiation) or a filtration media (e.g., leukofiltration) (e.g., to reduce the level of contaminating leukocytes).

In some embodiments of the aforementioned methods, the psoralen compound is a 4′-primary amino-substituted psoralen or 5′-primary amino-substituted psoralen. In some embodiments, the psoralen compound is a compound of formula I:

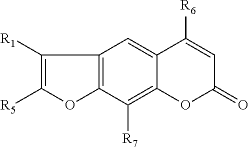

wherein in formula I, R₁ is
(CH₂)₂—NH₂,
—(CH₂)_{w}—R₂—(CH₂)_{z}—NH₂,
—(CH₂)_{w}—R₂—(CH₂)ₓ—R₃—(CH₂)_{z}—NH₂, or
(CH₂)_{w}—R₂—(CH₂)ₓ—R₃—(CH₂)_{y}—R₄—(CH₂)_{z}—NH₂;
   and R₂, R₃, and R₄ are independently O or NH, and w is a whole number from 1 to 5, x is a whole number from 2 to 5, y is a whole number from 2 to 5, and z is a whole number from 2 to 6; R₅, R₆ and R₇ are independently H or (CH₂)ᵥCH₃, and v is a whole number from 0 to 5. In some embodiments, the psoralen compound is 4′-(4-amino-2-aza)butyl-4,5′,8-trimethylpsoralen, 4′-(4-amino-2-oxa)butyl-4,5′,8-trimethylpsoralen, 4′-(2-aminoethyl)-4,5′,8-trimethylpsoralen, 4′-(5-amino-2-oxa)pentyl-4,5′,8-trimethylpsoralen, 4′-(5-amino-2-aza)pentyl-4,5′8-trimethylpsoralen, 4′-(6-amino-2-aza)hexyl-4,5′,8-trimethylpsoralen, 4′-(7-amino-2,5-oxa)heptyl-4,5′,8-trimethylpsoralen, 4′-(12-amino-8-aza-2,5-dioxa)dodecyl-4,5′,8-trimethylpsoralen, 4′-(13-amino-2-aza-6,11-dioxa)tridecyl-4,5′,8-trimethylpsoralen, 4′-(7-amino-2-aza)heptyl-4,5′,8-trimethylpsoralen, 4′-(7-amino-2-aza-5-oxa)heptyl-4,5′,8-trimethylpsoralen, 4′-(9-amino-2,6-diaza)nonyl-4,5′,8-trimethylpsoralen, 4′-(8-amino-5-aza-2-oxa)octyl-4,5′,8-trimethylpsoralen, 4′-(9-amino-5-aza-2-oxa)nonyl-4,5′,8-trimethylpsoralen, or 4′-(14-amino-2,6,11-triaza)tetradecyl-4,5′,8-trimethylpsoralen. In some embodiments, the psoralen compound is 4′-(4-amino-2-oxa)butyl-4,5′,8-trimethylpsoralen, which may also be referred to as 3-(2-Aminoethoxymethyl)-2,5,9-trimethyl-7H-furo[3,2-g]-1-benzopyran-7-one hydrochloride, or S-59, or amotosalen.

In some embodiments of the aforementioned methods, the method comprises pooling at least 3, at least 4, at least 5, at least 6, at least 7 or at least 8 platelet components. In some embodiments, the method comprises pooling 2 to 10 platelet components. In some embodiments, the method comprises pooling 3 to 10 platelet components. In some embodiments, the method comprises pooling 4 to 10 platelet components. In some embodiments, the method comprises pooling 4 to 9 platelet components. In some embodiments, the method comprises pooling 4 to 8 platelet components. In some embodiments, the method comprises pooling 4 to 7 platelet components. In some embodiments, the method comprises pooling 4 to 6 platelet components. In some embodiments, the method comprises pooling 3 platelet components, pooling 4 platelet components, pooling 5 platelet components, pooling 6 platelet components, pooling 7 platelet components or pooling 8 platelet components. In some embodiments the platelet components in the pooled platelet product are ABO blood group matched. In some embodiments the platelet components in the pooled platelet product are HLA matched.

In some embodiments of the aforementioned methods, the platelet components each comprise about 0.1×10¹¹ to about 2.2×10¹¹ platelets. In some embodiments, the platelet components each comprise about 0.4×10¹¹ to about 1.1×10 platelets. In some embodiments, the platelet components each comprise about 0.4×10¹¹ to about 0.8×10¹¹ platelets. In some embodiments, the platelet components each comprise about 0.6×10¹¹ to about 0.8×10¹¹ platelets. In some embodiments, the platelet components each comprise about 0.8×10¹¹ to about 1.1×10¹¹ platelets. In some embodiments, the platelet components each comprise about 0.8×10¹¹ platelets. In some embodiments, the platelet components each comprises about 0.9×10¹¹ platelets. In some embodiments, the platelet components each comprise about 1.0×10¹¹ platelets. In some embodiments, the platelet components each comprise about 1.1×10¹¹ platelets.

In some embodiments of the aforementioned methods, the transfusion ready platelet product comprises about 1.0×10¹¹ to about 8×10¹¹ platelets. In some embodiments, the transfusion ready platelet product comprises about 2.0×10¹¹ to about 8.0×10¹¹ platelets. In some embodiments, the transfusion ready platelet product comprises about 2.0×10¹¹ to about 7.0×10¹¹ platelets. In some embodiments, the transfusion ready platelet product comprises about 2.5×10¹¹ to about 7.0×10¹¹ platelets. In some embodiments, the transfusion ready platelet product comprises about 2.5×10¹¹ to about 6.0×10¹¹ platelets. In some embodiments, the transfusion ready platelet product comprises about 2.5×10¹¹ to about 5.0×10¹¹ platelets. In some embodiments, the transfusion ready platelet product comprises about 2.5×10¹¹ to about 4.0×10¹¹ platelets. In some embodiments, the transfusion ready platelet product comprises about 3.0×10¹¹ to about 7.0×10¹¹ platelets. In some embodiments, the transfusion ready platelet product comprises about 3.0×10¹¹ to about 6.0×10¹¹ platelets. In some embodiments, the transfusion ready platelet product comprises about 3.0×10¹¹ to about 5.0×10¹¹ platelets. In some embodiments, the transfusion ready platelet product comprises about 3.0×10¹¹ to about 4.0×10¹¹ platelets. In some embodiments, the transfusion ready platelet product comprises about 4.0×10¹¹ to about 6.4×10¹¹ platelets. In some embodiments, the transfusion ready platelet product comprises about 4.0×10¹¹ to about 7.0×10¹¹ platelets. In some embodiments, the transfusion ready platelet product comprises about 4.0×10¹¹ to about 6.0×10¹¹ platelets. In some embodiments, the transfusion ready platelet product comprises about 6.0×10¹¹ to about 7.0×10¹¹ platelets.

In some embodiments of the aforementioned methods, the transfusion ready platelet product further comprises donor plasma. In some embodiments, the platelet product comprises platelets in 100% plasma. In some embodiments of the aforementioned methods, the transfusion ready platelet product further comprises an additive solution. In some embodiments of the aforementioned methods, the transfusion ready platelet product comprises about 5 to 50% plasma and about 95 to 50% additive solution. In some embodiments, the transfusion ready platelet product comprises about 30-50% plasma and about 50-70% additive solution. In some embodiments, the transfusion ready platelet product comprises about 30%, about 35%, about 400, about 45% or about 50% plasma and the remainder as additive solution. In some embodiments, the transfusion ready platelet product comprises greater than 95% additive solution. In some embodiments, the additive solution is a platelet additive solution. In some embodiments of the aforementioned methods, the transfusion ready platelet product has not been subjected to treatment with a leukocyte reduction filter prior to administration to the subject. In some embodiments of the aforementioned methods, the transfusion ready platelet product comprises a platelet product in a storage container with suitable labeling for human use, wherein the labeling indicates that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the transfusion ready platelet product comprises a platelet product in a storage container and instructions for human use, wherein the instructions indicate that treatment with ionizing radiation (e.g., gamma irradiation, X-ray irradiation, X-irradiation) or a filtration medium (e.g., leukocyte filter) is not required prior to administration of the platelet product to a subject (e.g., human subject). In some embodiments, the labeling and/or instructions indicate that the transfusion ready platelet product has not been subjected to treatment with either ionizing radiation (e.g., gamma irradiation, X-ray irradiation, X-irradiation) or a filtration medium to reduce the level of contaminating leukocytes.

In some embodiments of the aforementioned methods, the method comprises administering the transfusion ready platelet product to the subject two or more times. In some embodiments, the method comprises administering an aforementioned platelet product to the subject 3 or more times, 4 or more times or 5 or more times. In some embodiments of the aforementioned methods, the method comprises administering two or more transfusions (e.g., units, doses) of the transfusion ready platelet product to the subject. In some embodiments, the method comprises administering three or more transfusions, 4 or more transfusions or 5 or more transfusions of the platelet product to the subject.

In some embodiments of the aforementioned methods, the subject has a platelet count of less than about 20,000/μL prior to administration of the platelet product. In some embodiments, the subject has a platelet count of less than about 10,000/μL prior to administration of the platelet product. In some embodiments, the subject has a platelet count of less than about 5,000/μL prior to administration of the platelet product. In some embodiments of the aforementioned methods, administering the platelet product to the subject results in a 1 hour corrected count increment (CCI) of greater than 5,000. In some embodiments, administering the platelet product to the subject results in a 1 hour corrected count increment (CCI) of greater than 10,000. In some embodiments, administering the platelet product to the subject results in a 1 hour corrected count increment (CCI) of greater than 15,000. In some embodiments, administering the platelet product to the subject results in a 1 hour corrected count increment (CCI) of greater than 20,000 or more. In some embodiments of the aforementioned methods, the leukocyte-related transfusion complication is transfusion-associated graft-versus-host disease (TA-GVHD). In some embodiments of the aforementioned methods, the leukocyte-related transfusion complication is alloimmunization. In some embodiments, the leukocyte-related transfusion complication is alloimmune platelet refractoriness. In some embodiments of the aforementioned methods, the leukocyte-related transfusion complication is cytokine stimulation and/or production. In some embodiments of the aforementioned methods, the leukocyte-related transfusion complication is microchimerism. In some embodiments of the aforementioned methods, the leukocyte-related transfusion complication is a febrile non-hemolytic transfusion reaction (FNHTR).

In another aspect, the present disclosure provides a transfusion ready platelet product for use in treating a subject in need thereof (e.g., in need of a platelet product), comprising one or more whole blood-derived platelet components in a storage container with suitable labeling for human use, wherein the one or more platelet component(s) have been subjected to photochemical treatment with a psoralen compound to inactivate pathogens and leukocytes, if present, and wherein the one or more platelet component(s) have not been subjected to treatment with ionizing radiation (e.g., gamma irradiation, X-ray irradiation, X-irradiation) or a filtration media (e.g., leukofiltration) (e.g., to reduce the level of contaminating leukocytes), wherein the transfusion ready platelet product comprises suitable labeling and/or accompanying instructions indicating that a further step of treatment with either ionizing radiation (e.g., gamma irradiation, X-ray irradiation, X-irradiation) or a filtration medium (e.g., leukofiltration) is not required for administration to a subject. In some embodiments, the transfusion ready platelet product further comprises suitable labeling and/or accompanying instructions indicating that the transfusion ready platelet product has not been subjected to treatment with either ionizing radiation (e.g., gamma irradiation, X-ray irradiation, X-irradiation) or a filtration medium (e.g., leukofiltration). In some embodiments, the one or more whole blood-derived platelet components are prepared by a buffy coat method. In some embodiments, the one or more whole blood-derived platelet components are prepared by a platelet rich plasma (PRP) method. In some embodiments, the transfusion ready platelet product is a platelet product for use in treating a subject with elevated risk for a leukocyte-related transfusion complication.

In some embodiments, the storage container comprises labeling indicating that treatment with either ionizing radiation or a leukocyte reduction filter is not required for administration to a subject. In some embodiments, the transfusion ready platelet product comprises at least 3, at least 4, at least 5, at least 6, at least 7 or at least 8 platelet components. In some embodiments, the transfusion ready platelet product comprises 2 to 10 platelet components, 3 to 10 platelet components, 4 to 10 platelet components, 4 to 9 platelet components, 4 to 8 platelet components, 4 to 7 platelet components, 4 to 6 platelet components, 3 platelet components, 4 platelet components, 5 platelet components, 6 platelet components, 7 platelet components or 8 platelet components. In some embodiments the platelet components are ABO blood group matched. In some embodiments the platelet components are HLA matched.

In some embodiments, the transfusion ready platelet product comprises about 1.0×10¹¹ to about 8×10¹¹ platelets, about 2.0×10¹¹ to about 8.0×10¹¹ platelets, about 2.0×10¹¹ to about 7.0×10¹¹ platelets, about 2.5×10¹¹ to about 7.0×10 platelets, about 2.5×10¹¹ to about 6.0×10¹¹ platelets, about 2.5×10¹¹ to about 5.0×10¹¹ platelets, about 2.5×10¹¹ to about 4×10¹¹ platelets, about 3.0×10¹¹ to about 7.0×10¹¹ platelets, about 3.0×10¹¹ to about 6.0×10¹¹ platelets, about 3.0×10¹¹ to about 5.0×10¹¹ platelets, about 3.0×10¹¹ to about 4.0×10¹¹ platelets, about 4.0×10¹¹ to about 6.4×10¹¹ platelets, about 4.0×10¹¹ to about 7.0×10 platelets, about 4.0×10 to about 6.0×10¹¹ platelets, or about 6.0×10¹¹ to about 7.0×10¹¹ platelets. In some embodiments, the transfusion ready platelet product further comprises donor plasma. In some embodiments, the transfusion ready platelet product further comprises an additive solution. In some embodiments, the transfusion ready platelet product comprises about 5 to 50% plasma and about 95 to 50% additive solution. In some embodiments, the transfusion ready platelet product is a platelet product for use in treating a subject with elevated risk for a leukocyte-related transfusion complication. In some embodiments, the transfusion ready platelet product is a platelet product for use in reducing leukocyte-related transfusion complications in a recipient of a platelet product.

In another aspect, the present disclosure sets forth a method of treating a subject in need thereof with a platelet product, comprising administering to a human subject a transfusion ready platelet product as provided herein. In another aspect, the present disclosure sets forth a method of providing a platelet product to a subject in need thereof, comprising administering to a human subject a transfusion ready platelet product as provided herein. In some embodiments, the subject is a subject with elevated risk for a leukocyte-related transfusion complication. In some embodiments, the method comprises administering the transfusion ready platelet product 2 or more times, 3 or more times, 4 or more times or 5 or more times In some embodiments, the subject is a human subject with elevated risk for a leukocyte-related transfusion complication. In some embodiments, the method comprises administering the transfusion ready platelet product to the subject 2 or more times. In some embodiments, the method comprises administering the transfusion ready platelet product to the subject 3 or more times, 4 or more times or 5 or more times. In some embodiments, the method comprises administering two or more transfusions (e.g., units, doses) of the transfusion ready platelet product to the subject. In some embodiments, the method comprises administering three or more transfusions, 4 or more transfusions or 5 or more transfusions of the transfusion ready platelet product to the subject.

In some embodiments of the aforementioned methods, photochemical treatment with a psoralen compound inactivates at least about 4 logs, at least about 5 logs, at least about 6 logs or at least about 7 logs or more of leukocytes. In some embodiments of the aforementioned methods, photochemical treatment with a psoralen compound decreases the amount of contaminating leukocytes in a platelet preparation to less than about 5×10⁶, less than about 4×10⁶, less than about 3×10⁶, less than about 2×10⁶, less than about 1×10⁶, less than about 9×10, less than about 8×10′, less than about 7×10⁵, less than about 6×10⁵, or less than about 5×10⁵ leukocytes per platelet preparation. In some embodiments, the amount of contaminating leukocytes is determined by limiting dilution analysis.

### DETAILED DESCRIPTION

The term “platelet product” or “platelet-containing product” means any blood derived product comprising platelets as the primary therapeutic component. Platelets are cells necessary for the coagulation of blood, and such platelet-containing products as described herein may be transfused to patients in need thereof, such as for patients with bleeding due to thrombocytopenia, platelet dysfunction, or while undergoing surgery. Such platelet products can be further comprised of blood plasma, anticoagulant solution used during collection, and alternatively, or in addition, a suitable storage solution, such as an additive solution, such as for example a platelet additive solution. Since other cells present in the donated blood are not completely removed, platelet-containing products will also contain some levels of red blood cells and white blood cells, along with plasma components. As the cellular components of the platelet product are generally a very small percentage of the volume, as is any anticoagulant solution, the platelet product is typically referred to by the composition of plasma and any platelet additive solution (PAS), if present. Worldwide the collecting and storage of platelet products varies, in that the platelets may be collected and stored in various volumes and amounts, as well as in various media, ranging from essentially 100% plasma to a percentage of plasma and a percentage of a suitable storage media, such as for example 35% plasma and 65% suitable PAS.

A “pooled platelet product”, as used herein refers to a platelet product prepared from platelets obtained from more than one whole blood donation and subsequently combined (e.g., in a single container) prior to final product use, such as before transfusion. Generally, the whole blood donations are obtained from different donors. Platelets may be pooled at any stage after whole blood donation and prior to final product use, including but not limited to pooling as a platelet component (e.g., platelet concentrate), before or after any addition of additive solution, before or after any storage period, and before or after any pathogen inactivation treatment or processing.

A “pathogen inactivated platelet product” as used herein describes a platelet product that has undergone processing (e.g., by the methods described herein) to inactivate pathogens, and leukocytes, that may be present in the platelet product (e.g., unit of platelets, pooled platelet product), where it is understood that the process does not necessarily inactivate completely all pathogens and leukocytes that may be present, but substantially reduces the amount of pathogens and leukocytes to significantly reduce the risk of a transfusion associated disease. The inactivation of a pathogen may be assayed by measuring the number of infective pathogen (e.g., viral or bacterial particles) in a certain volume, and the level of inactivation is typically represented in the log reduction in the infectivity of the pathogen, or log reduction in titer. Methods of assaying log reduction in titer, and measurements thereof for pathogen inactivation are known in the art. When the inactivation process is tested against a variety of pathogens, the reduction in a particular active pathogen is at least about 1 log, at least about 2 log, at least about 3 log, at least about 4 log, or at least about 5 log reduction in titer. The inactivation of leukocytes may be assayed, for example, using T-cell clonal expansion assay, expression of activation antigens and mixed lymphocyte culture assay, and/or inhibition of cytokine synthesis (Corash et al., 2004, Bone Marrow Transplantation 33:1-7). The inactivation of leukocytes may be, for example, at least about 1 log, at least about 2 log, at least about 3 log, at least about 4 log, or at least about 5 log reduction or more.

A “transfusion ready platelet product” as used herein refers to a platelet product in a storage container (e.g., blood product bag) with suitable labeling for human use, which requires no further processing or treatment of the platelet contents therein prior to administration to a subject. Such transfusion ready platelet products comprise platelets that have been subjected to a pathogen inactivation processing step (e.g., photochemical treatment, such as with a psoralen compound, to inactivate pathogens and leukocytes, if present) during preparation, as provided in the present disclosure, and may exclude platelets that have been subjected to one or more other specified treatment or processing steps (e.g., ionizing radiation such as gamma irradiation, filtration media such as leukofiltration, to reduce the level of contaminating leukocytes) as set forth in the present disclosure. The transfusion ready platelet product may be produced from an individual platelet unit or more than one unit (e.g., a pooled platelet product) and may be tested for one or more quality measures (e.g., bacterial testing, pH, platelet integrity assessed by platelet count and supernatant LDH, lactate concentration, platelet content, platelet morphology score, glucose concentration, platelet aggregation, extracellular adenosine triphosphate concentration, total adenosine triphosphate concentration, extent of shape change, and platelet hypotonic shock response). It is understood that storage of a transfusion ready platelet product may require agitation (e.g., shaking) under controlled conditions (e.g., controlled temperature, controlled humidity) and that such storage requirements are not considered to be further processing or treatment of the platelet contents therein. In certain embodiments, a transfusion ready platelet product of the present disclosure is suitable for administration to subjects with elevated risk for a leukocyte-mediated transfusion complication.

The term “platelet additive solution” means any suitable aqueous composition that can be used in the storage of a platelet product. Such platelet additive solutions typically provide nutrients and buffering capacity to allow for extended storage of platelets while maintaining suitable platelet function. A variety of suitable platelet additive solutions may be used in the storage of platelets, where such solutions can be added to a unit of platelets in various amounts, such that a unit of platelets may comprise anywhere from about 0 to 95% platelet additive solution, about 5 to 95% platelet additive solution, about 50 to 95% platelet additive solution, about 50 to 75% platelet additive solution, about 53% to 700/% platelet additive solution, or about 53 to 68% platelet additive solution. For example, platelets may be stored in about 65% platelet additive solution and about 35% plasma, providing a unit of platelets comprising platelets, about 65% platelet additive solution, and about 35% plasma. Typically, in the methods described herein, a unit of platelets will be prepared to a desired level of plasma by addition of the platelet additive solution, either automatically in apheresis collection, or manually in the processing of platelet rich plasma or buffy coat platelets. Platelet additives for use herein are described in terms of their aqueous concentration of components prior to their addition to the platelets to give the desired level of plasma in the additive containing unit of platelets. Thus, for a platelet additive solution described as having, for example, an acetate concentration of X μM, when used to provide a platelet unit at 65% platelet additive solution and 35% plasma, will have an acetate concentration of 0.65X μM (not including any amount of acetate that may be present in the plasma). Platelet additive solutions typically include sodium chloride and one or more components selected from the group consisting of citrate, phosphate, acetate, magnesium, potassium, calcium, gluconate, glucose, and bicarbonate. The following examples comprise sodium chloride and the indicated components: PAS-A (also referred to as PAS(1)) comprising citrate, phosphate and potassium; PAS-B (also referred to as PAS II, PAS-2, SSP, or T-Sol) comprising citrate and acetate: PAS-C (also referred to as PAS III, PAS-3, or Intersol) comprising citrate, phosphate, and acetate; PAS-D (also referred to as Composol) comprising citrate, phosphate, acetate, magnesium, potassium, and gluconate; PAS-E (also referred to as PAS IIIM or SSP+) comprising citrate, phosphate, acetate, magnesium, and potassium; PAS-F (also referred to as PlasmaLyte A) comprising acetate, magnesium, potassium, and gluconate; PAS-G comprising citrate, phosphate, acetate, magnesium, potassium, and glucose; InterSol-G (also referred to as PAS IV) comprising citrate, phosphate, acetate, magnesium, potassium, calcium and glucose; Isoplate (also referred to as Isolyte S) comprising phosphate, acetate, magnesium, potassium, and gluconate; PAS V comprising citrate, acetate, phosphate, magnesium, potassium, calcium, glucose, and bicarbonate; and M-Sol comprising citrate, acetate, magnesium, potassium, calcium, glucose and bicarbonate. Detailed composition of these platelet additive solutions is found in the following Tables 1a and 1b. Preferred platelet additive solutions for use in the examples described herein include, for example, SSP+(PAS-E) and InterSol (PAS-C). The composition of SSP+ is 69 mM sodium chloride, 30 mM sodium acetate, 10 mM trisodium citrate, 26 mM sodium phosphate, 5 mM potassium chloride, and 1.5 mM magnesium chloride or magnesium sulfate. The composition of InterSol is 77 mM sodium chloride, 33 mM sodium acetate, 11 mM trisodium citrate, and 28 mM sodium phosphate.

TABLES 1a and 1b
composition of platelet additive solutions.

| | Platelet additive solution component concentration (mM) | | | |
|---|---|---|---|---|
| Component | PAS-B | PAS-C | PAS-D | PAS-E |
| NaCl | 116 | 77 | 90 | 69 |
| Acetate | 30 | 33 | 27 | 30 |
| Citrate | 10 | 11 | 11 | 10 |
| Phosphate | 0 | 28 | 0 | 26 |
| Gluconate | 0 | 0 | 23 | 0 |
| K⁺ | 0 | 0 | 5 | 5 |
| Mg²⁺ | 0 | 0 | 1.5 | 1.5 |

| | Platelet additive solution component concentration (mM) | | | | |
|---|---|---|---|---|---|
| Component | PAS-F | Isoplate | InterSol-G | PAS V | M-Sol |
| NaCl | 90 | 92.7 | 69.8 | 69.4 | 77 |
| Acetate | 27 | 27.2 | 30 | 30 | 21 |
| Citrate | 0 | 0 | 10 | 10 | 9.4 |
| Phosphate | 0 | 0.5 | 9.6 | 9.4 | 0 |
| Gluconate | 23 | 22.9 | 0 | 0 | 0 |
| K⁺ | 5 | 5 | 5 | 5 | 3 |
| Mg²⁺ | 3 | 1.5 | 1.5 | 1.5 | 1.6 |
| Ca²⁺ | 0 | 0 | 1 | 1 | 1 |
| Glucose | 0 | 0 | 18.5 | 16.8 | 15 |
| Bicarbonate | 0 | 0 | 0 | 9 | 43.5 |

The term “pathogen inactivation” or “photochemical treatment with a psoralen compound” means treatment with any suitable psoralen compound under conditions that can inactivate a pathogen and/or a leukocyte that may be present in a platelet-containing product. A suitable psoralen compound requires some level of light in order to sufficiently inactivate a pathogen and/or a leukocyte. For example psoralens or a psoralen generally describes the psoralen core compound and any derivative thereof (e.g. amotosalen). Such derivatives comprise the core compound structure as well as additional substituents on the core. For example, psoralen derivatives may be derived from substitution of the linear furocoumarin at the 3, 4, 5, 8, 4′, or 5′ positions. Descriptions of such compounds include any salts thereof. Exemplary psoralens for use in the photochemical treatment methods described herein are illustrated below, and include for example 4′-(4-amino-2-oxa)butyl-4,5′,8-trimethyl psoralen, which may also be referred to as 3-[(2-aminoethoxy)methyl]-2,5,9-trimethyl-7H-furo[3,2-G][1]benzopyran-7-one-hydrochloride, 3-(2-aminoethoxymethyl)-2,5,9-trimethylfuro[3,2-g]chromen-7-one, S-59, amotosalen and any salts thereof. Treatment or processing (e.g., photochemical treatment) of platelets with psoralen (e.g., amotosalen) inactivation refers to combining platelets (e.g., unit of platelets, platelet component, pooled platelet product) with the psoralen and illuminating with a suitable dose of UVA light in order to inactivate pathogens and/or leukocytes that may be present in the platelets. In some embodiments, amotosalen inactivated platelets are pathogen inactivated according to commercial methods for platelets, or by similar methods. Such methods provide, for example, a platelet unit prior to addition of amotosalen having a volume within the range of 255 to 420 mL, a total platelet content in this volume of 2.5 to 8.0×10¹¹, where the platelet unit is in 100% plasma, or 32 to 47% plasma with the remaining volume platelet additive solution (i.e. 53 to 68% PAS, where % plasma+% PAS=100), which is combined with either 15 mL or 17.5 mL of a 3 mM amotosalen solution, resulting in a platelet unit ready for illumination having an amotosalen concentration within the range of about 120 to 193 μM prior to illumination. The resulting solution is illuminated with the equivalent of about 3 J/cm² of light in the UVA wavelength range. While the methods described herein are applied to platelet units using known systems such as the use of amotosalen for pathogen inactivation in platelets, they are applicable to any pathogen inactivated unit of platelets. It may be desirable to remove the psoralen and associated photoproducts after treatment and such removal methods are known in the art.

### Platelet Preparation

Whole blood for use in the preparation of platelets (e.g., buffy coat, PRP) as described herein may be collected by a variety of procedures known in the art. Generally, whole blood may be obtained by “manual” collection from healthy donors. As commonly understood and as used herein, manual collection refers to a collection method where whole blood is allowed to drain from the donor and into a collection container without the use of external pumps or similar devices. This is in contrast to so-called automated procedures where blood is withdrawn from a donor and further processed by an automated instrument that typically includes a processing or separation device and pumps for moving blood or blood components into and out of the device.

Withdrawing blood from a donor typically includes inserting a needle into the donor's arm (and, more specifically, the donor's vein) and withdrawing blood from the donor through the needle. The “venipuncture” needle typically has attached to it one end of a plastic tube that provides a flow path for the blood. The other end of the plastic tube terminates in one or more pre-attached plastic blood containers (e.g., bags) for collecting the blood. The needle, tubing and containers make up a blood collection set which is pre-sterilized and disposed of after a single use. It may be desirable for collection of whole blood to be completed relatively quickly, such as for example, with a whole blood donation time of less than about 15 minutes, or less than about 12 minutes, or less than about 10 minutes. Whole blood collection volumes may vary, for example 405-495 mL for 450 mL collection containers or 450-550 mL for 500 mL collection containers. The sterile blood collection container typically serves as the primary container for initial separation of platelets in the buffy coat or PRP methods. The blood collection container and plastic tubing may also include a volume of a liquid anticoagulant. Anticoagulant is required because of the tendency of blood to clot and adhere to the walls of the plastic surfaces. Exemplary anticoagulants are well known in the art and may include, but are not limited to, an anticoagulant citrate phosphate dextrose (CPD) solution, an anticoagulant citrate phosphate double dextrose (CP2D) solution, an anticoagulant citrate phosphate dextrose adenine (CPDA) solution (e.g., CPDA-1), an acid citrate dextrose (ACD) solution (e.g., ACD-A), and an anticoagulant sodium citrate 4% w/v solution.

Blood may, if desired, be identified or characterized with respect to one or more parameters, such as for example, hematocrit, hemoglobin, donor gender, whole blood volume, packed cell volume and/or platelet count. In addition, at or near the time of collection and prior to transfusion to a patient, tests may be performed for determining blood type and the presence of pathogens such as virus, bacteria and/or other foreign substances in the donor's blood. Such testing generally requires obtaining a sample of the donor's blood, without compromising the sterility of the system and/or the collected blood product.

Whole blood units from donors may be processed immediately after collection, or may be stored for a period of time after donation (e.g., resting period), prior to subjecting the blood to processing for production of platelets (e.g., whole blood-derived platelet components). For example, a whole blood unit may be stored for at least 1 hour, at least 2 hr, at least 3 hr, at least 4 hr, at least 5 hr, at least 6 hr, at least 7 hr, at least 8 hr or more, or overnight. Preferably, the whole blood unit is stored for less than 24 hours. In some embodiments, the whole blood unit is stored for less than 8 hours. In some embodiments, the whole blood unit is stored for at least 8 hr and less than 24 hr. The whole blood unit may also be cooled from its initial post-collection temperature, for storage and/or processing (e.g., at a controlled temperature). The whole blood unit may be cooled, for example, to room temperature, to 18-25° C., to 20-24° C., or to about 22° C. following donation.

For preparation of platelets (e.g., whole-blood derived platelets) by the buffy coat method, whole blood is centrifuged under conditions to separate the components into a lower red blood cell layer, a middle buffy coat layer containing the platelets and an upper platelet poor plasma layer. Buffy coat production methods for platelets are well known in the art. Centrifugation conditions may be optimized according to blood center procedures, available centrifugation equipment, etc., but generally the initial centrifugation may be performed, for example, for 7 min. at 5000×g at 22° C., or 5 min. The buffy coat is isolated, for example, by removing (e.g., expressing) the upper plasma layer and the lower red cell layer, leaving the buffy coat in the collection container. The isolated buffy coats may be further processed, for example, by pooling (e.g., 4-6 buffy coats) and/or addition of additive solution (PAS) and/or plasma to achieve a desired volume and platelet concentration (e.g., within pathogen inactivation processing criteria), followed by a lower speed “soft” centrifugation to separate the platelets from white blood cells. Such a lower speed centrifugation may be performed for example, for 3 min. at 2000×g at 22° C. or 8 min. at 500×g at 22° C., followed by expressing the platelet suspension into a storage bag. The platelet components are stored in a platelet incubator with agitation at, for example, about 22° C., until further processing (e.g., pooling, pathogen inactivation treatment).

For preparation of platelets (e.g., whole blood-derived platelets) by the PRP method, whole blood is centrifuged under conditions to separate the components into a lower red blood cell layer containing white blood cells and an upper platelet rich plasma (PRP) layer. PRP production methods for platelets are well known in the art. Centrifugation conditions may be optimized according to blood center procedures, available centrifugation equipment, etc., but generally the initial centrifugation is performed as a lower speed “soft spin”, for example, for 3 min. at 2000×g at 22° C. Platelet rich plasma is separated from the red blood cell layer by expressing the upper PRP layer (e.g., using a Compomat G-5), followed by a secondary, faster speed “hard” centrifugation of the PRP to separate the platelets from the plasma. Such a hard spin may be performed for example, for 5 min. at 5000×g at 22° C., followed by removal of plasma from the platelet concentrate, leaving a desired volume of plasma for resuspension of the platelet component. The isolated PRP derived platelet components may be further processed, for example, by pooling (e.g., 4-6 PC) and/or addition of additive solution (PAS) and/or plasma to achieve a desired volume and platelet concentration (e.g., within pathogen inactivation processing criteria). Platelets are stored in a platelet incubator with agitation, at for example, about 22° C., until further processing (e.g., pooling, pathogen inactivation treatment).

A variety of desired volumes and platelet amounts (e.g., total platelets, platelet concentration) may be achieved, for example, based on volume of plasma and/or PAS included with the platelets, the number of platelet components pooled and the volume of the starting whole blood donation. For example, a platelet component may comprise about 0.1×10¹¹ to about 2.2×10¹¹ platelets, about 0.4×10¹¹ to about 1.1×10¹¹ platelets, about 0.4×10¹¹ to about 0.8×10¹¹ platelets, about 0.6×10¹¹ to about 0.8×10¹¹ platelets, about 0.8×10¹¹ to about 1.1×10¹¹ platelets, about 0.8×10¹¹ platelets, about 0.9×10¹¹ platelets, about 1.0×10¹¹ platelets or about 1×10¹¹ platelets. Two or more platelet components may be pooled (e.g., mixed, combined) and such pooling may provide a platelet product that comprises, for example, at least 3, at least 4, at least 5, at least 6, at least 7 or at least 8 platelet components, 2 to 10 platelet components, 3 to 10 platelet components, 4 to 10 platelet components, 4 to 9 platelet components, 4 to 8 platelet components, 4 to 7 platelet components, 4 to 6 platelet components, 3 platelet components, 4 platelet components, 5 platelet components, 6 platelet components, 7 platelet components or 8 platelet components. In some embodiments the pooled platelet components are ABO blood group matched and/or HLA matched. In some embodiments, pooled platelet components may be subsequently split into two or more platelet products (e.g. transfusion ready platelet product) each comprising a desired amount of platelets, such as, for example, an amount of platelets for a therapeutic dose. Platelet products (e.g., pooled platelet products, transfusion ready platelet products) may comprise, for example, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets, at least about 3.0×10¹¹ platelets, at least about 3.5×10¹¹ platelets, at least about 4.0×10¹¹ platelets, about 1.0×10¹¹ to about 8×10¹¹ platelets, about 2.0×10¹¹ to about 8.0×10¹¹ platelets, about 2.0×10¹¹ to about 7.0×10¹¹ platelets, about 2.5×10¹¹ to about 7.0×10¹¹ platelets, about 2.5×10¹¹ to about 6.0×10¹¹ platelets, about 2.5×10¹¹ to about 5.0×10¹¹ platelets, about 2.5×10¹¹ to about 4.0×10¹¹ platelets, about 3.0×10¹¹ to about 7.0×10¹¹ platelets, about 3.0×10¹¹ to about 6.0×10¹¹ platelets, about 3.0×10¹¹ to about 5.0×10¹¹ platelets, about 3.0×10¹¹ to about 4.0×10¹¹ platelets, about 4.0×10¹¹ to about 6.4×10¹¹ platelets, about 4.0×10¹¹ to about 7.0×10¹¹ platelets, about 4.0×10¹¹ to about 6.0×10¹¹ platelets or about 6.0×10¹¹ to about 7.0×10¹¹ platelets. Platelets (e.g., platelet component, platelet product, pooled platelet product, transfusion ready platelet product) may also include plasma and/or additive solution, for example, about 5 to 50% plasma and about 95 to 50%° additive solution, about 30-50% plasma and about 50-70% additive solution, about 30%, about 35%, about 40%, about 45% or about 50% plasma and the remainder as additive solution, or greater than 95% additive solution. In some embodiments, the additive solution is a platelet additive solution. In some embodiments, the platelet product comprises platelets in 100% plasma.

Also described in the disclosure are platelet products (e.g., transfusion ready platelet products), such as platelet product provided to a subject in need thereof, whereby such platelet products have not been subjected to treatment with either ionizing radiation (e.g., gamma irradiation, X-ray irradiation, X-irradiation) or a filtration medium (e.g., a leukocyte reduction filter) to reduce the level of contaminating leukocytes. The disclosure contemplates that such platelet products have not been subjected to such treatment or filtration during preparation of the platelet product (e.g., as whole blood, as platelet component, as platelet concentrate) or after preparation of the platelet product (e.g., pooled platelet product, transfusion ready platelet product). Such a platelet product would be administered to a subject in need thereof without any treatment with ionizing radiation or filtration medium to reduce the level of contaminating leukocytes prior to administration to a subject. Filtration medium to reduce the level of contaminating leukocytes in a blood product (e.g., leukocyte reduction filter) refer to filtration medium intended to be used for such a reduction (e.g., removal) purpose and are well known in the field of blood collection and transfusion medicine, and multiple examples of such filtration medium are commercially available, such as for example as leukoreduction filters (e.g., IUMGARD®, Terumo BCT). Generally such a filtration medium has received approval by one or more governmental or regulatory authorities for use in the intended purpose of such leukocyte reduction for whole blood or a blood component such as for example, platelets.

In some embodiments of the aforementioned methods, the method further comprises storing the platelet component (e.g., prior to pooling) for a period of about 2 hours to about 5 days, about 1 hour, about 2 hours, about 3 hours, about 4 hours, about 5 hours, about 6 hours, about 7 hours, about 8 hours, about 10 hours, about 12 hours, about 14 hours, about 16 hours, about 18 hours, about 20 hours, about 22 hours or about 24 hours. In some embodiments, such storage is for about 1 day, about 2 days, about 3 days, about 4 days or about 5 days. Generally the platelet component is stored with agitation.

### Psoralen Compounds

A variety of psoralen compounds (e.g., psoralen derived compounds, psoralen derivatives, salts thereof) are contemplated for use in photochemical treatment of platelets in the methods disclosed herein, such as for example, the psoralen compounds described in U.S. Pat. No. 5,593,823. Non-limiting examples of psoralen compounds may include, for example, psoralen compounds that are a 4′-primary amino-substituted psoralen or 5′-primary amino-substituted psoralen. Preferred psoralen compounds include, for example, psoralen compounds of the following formula I:

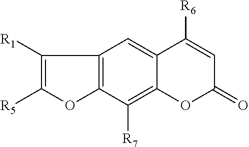

wherein R₁ is
(CH₂)₂—NH₂,
—(CH₂)_{w}—R₂—(CH₂)_{z}—NH₂,
—(CH₂)_{w}—R₂—(CH₂)ₓ—R₃—(CH₂)_{z}—NH₂, or
(CH₂)_{w}—R₂—(CH₂)ₓ—R₃—(CH₂)_{y}—R₄—(CH₂)_{z}—NH₂;
   and R₂, R₃, and R₄ are independently O or NH, and w is a whole number from 1 to 5, x is a whole number from 2 to 5, y is a whole number from 2 to 5, and z is a whole number from 2 to 6; R₅, R₆ and R— are independently H or (CH₂)ᵥCH₃, and v is a whole number from 0 to 5.

Exemplary psoralen compounds for use as disclosed herein, include for example, 4′-(4-amino-2-aza)butyl-4,5′,8-trimethylpsoralen, 4′-(4-amino-2-oxa)butyl-4,5′,8-trimethylpsoralen, 4′-(2-aminoethyl)-4,5′,8-trimethylpsoralen, 4′-(5-amino-2-oxa)pentyl-4,5′,8-trimethylpsoralen, 4′-(5-amino-2-aza)pentyl-4,5′8-trimethylpsoralen, 4′-(6-amino-2-aza)hexyl-4,5′,8-trimethylpsoralen, 4′-(7-amino-2,5-oxa)heptyl-4,5′,8-trimethylpsoralen, 4′-(12-amino-8-aza-2,5-dioxa)dodecyl-4,5′,8-trimethylpsoralen, 4′-(13-amino-2-aza-6,11-dioxa)tridecyl-4,5′,8-trimethylpsoralen, 4′-(7-amino-2-aza)heptyl-4,5′,8-trimethylpsoralen, 4′-(7-amino-2-aza-5-oxa)heptyl-4,5′,8-trimethylpsoralen, 4′-(9-amino-2,6-diaza)nonyl-4,5′,8-trimethylpsoralen, 4′-(8-amino-5-aza-2-oxa)octyl-4,5′,8-trimethylpsoralen, 4′-(9-amino-5-aza-2-oxa)nonyl-4,5′,8-trimethylpsoralen, or 4′-(14-amino-2,6,11-triaza)tetradecyl-4,5′,8-trimethylpsoralen. One particularly preferred psoralen compound for use as disclosed herein is 4′-(4-amino-2-oxa) butyl-4,5′,8-trimethylpsoralen, which is also known as 3-(2-Aminoethoxymethyl)-2,5,9-trimethyl-7H-furo[3,2-g]-1-benzopyran-7-one hydrochloride, or S-59, or amotosalen. Amotosalen is the psoralen compound available commercially in the INTERCEPT™ Blood System for pathogen reduction of platelets and plasma.

### Platelet Characterization

Whole blood derived platelets (e.g., PC, pooled concentrates) prepared using the methods herein may be characterized by a variety of qualitative and/or quantitative methods known in the art. Such characterization may include, for example, platelet count, red and/or white blood cell amount (e.g., contamination), lipid contamination, platelet aggregation, platelet recovery, platelet viability, swirling pattern, potency, platelet survival, morphology, functional activity, activation markers, blood gas (pO2, pCO2), pH, glucose, lactate, volume, concentration of growth factors and icterus.

Contamination of platelets with RBC may be determined by any of several methods known in the art, such as for example, visual inspection for color indicative of RBC contamination. More specifically, RBC contamination above a certain level (e.g., >400 RBC/mL), results in platelets that exhibit discoloration from a light pink/salmon, reddish-orange color tinge to a marked red discoloration, which may be compared to standard visual inspection charts. Lipid contamination (e.g., lipemia) may similarly be determined by visual methods, with increased opacity, ‘milky’ white appearance, large lipid particles that include lipoproteins and chylomicrons, and the like. Platelet aggregation may be determined visually and/or using any of a number of techniques and devices, such as for example, platelet aggregometry, optical aggregometers, lumi-aggregometers, light transmission aggregometry or turbidometric aggregometry. White blood cell contamination may be determined by counting, for example manually performing a leukocyte count (e.g., using a Neubauer counting chamber). Platelet morphology may be visually inspected at different levels of resolution, including for example, with a discs vs. spheres estimate, and the presence of different morphological forms may be quantitated. Functionality can be estimated, for example, by platelet response to osmotic stress and by the extent of agonist-induced shape change. Aggregation to increasing concentrations of physiologic agonists such as ADP, collagen, epinephrine, or to dual agonist combinations of ADP/epinephrine and ADP/collagen will give an idea of the responsiveness of the platelet. Volume may be determined by weight and using a factor, such as 1.01 g/ml, as the specific gravity of platelets in additive solution. Platelet serotonin uptake and agonist-induced serotonin secretion and agonist-induced expression of platelet activation markers such as GMP-140, will also evaluate the platelet physiologic response. Additionally, platelet cellular levels of ATP, glucose, and lactate provide an indication of platelet performance. Activation of platelets is associated with surface expression of the various surface antigens, such as for example, GMP-140 (P-selectin, CD 62), CD 63, and the active form (fibrinogen-binding) of GPIIb/IIIa (detected by PAC-1). Thromboglobulin and/or Platelet Factor 4 released by activated platelets into the medium are platelet-specific proteins and can be measured as indicators of platelet activation. Platelet Factor 3 activity (procoagulant surface for binding clotting proteins) also increases with platelet activation. Assays are also commercially available to perform such characterization of platelets in many cases.

### Thrombocytopenia and Platelet Dysfunction

The methods disclosed herein relate to providing a platelet product (e.g., transfusion ready platelet product) to a subject in need thereof. Generally, a subject in need of a platelet product is a subject, such as for example, a human patient, that is exhibiting low platelet count (thrombocytopenia) and/or platelet dysfunction at a level of severity such that administration (e.g., transfusion, infusion) of donor platelets is indicated by accepted medical practice, or who, because of a current disease or condition, and/or anticipated course of medical treatment (e.g., chemotherapy, radiation therapy), is expected to exhibit thrombocytopenia at a severity level where administration of donor platelets is appropriate.

Thrombocytopenia may be characterized into three major causes of low platelets: insufficient generation of platelets by the bone marrow, increased breakdown of platelets in the bloodstream, and increased breakdown of platelets in the spleen or liver. Some examples of conditions that may exhibit insufficient generation of platelets by bone marrow (e.g., Diminished or absent megakaryocytes in bone marrow) include, for example, aplastic anemia, cancer of the bone marrow (e.g., leukemia), cirrhosis, folate deficiency, infections in the bone marrow, myelodysplastic syndrome (e.g., bone marrow does not make enough blood cells or makes defective cells), vitamin B12 deficiency, use of certain drugs (e.g., chemotherapy treatment, myelosuppressive drugs, hydroxyurea, interferon alfa-2b), Botezomib use, and HIV-associated thrombocytopenia. Some examples of conditions that may cause increased breakdown of platelets include, for example, disseminated intravascular coagulation (DIC), drug-induced nonimmune thrombocytopenia, drug-induced immune thrombocytopenia, hypersplenism, immune thrombocytopenic purpura (ITP) and thrombotic thrombocytopenic purpura. Some examples of conditions related to increased breakdown of platelets in the spleen or liver may include, for example, cirrhosis with congestive splenomegaly, Gaucher disease and myelofibrosis with myeloid metaplasia. Destruction of platelets may, for example, be immunologic-mediated or nonimmunologic-mediated. Some examples of immunologic destruction of platelets may include, for example, connective tissue disorders, drug-induced thrombocytopenia, HIV-associated thrombocytopenia, immune thrombocytopenia, lymphoproliferative disorders, neonatal alloimmune thrombocytopenia, posttransfusion purpura. Some examples of nonimmunologic destruction may include, for example, certain systemic infections (e.g., Epstein-Barr virus, cytomegalovirus), disseminated intravascular coagulation, pregnancy (gestational thrombocytopenia), sepsis, thrombocytopenia in acute respiratory distress syndrome, and thrombotic thrombocytopenic purpura-hemolytic-uremic syndrome. Additionally, dilution by red blood cell replacement or exchange transfusion may result in thrombocytopenia.

Clinical indication for administration (e.g., transfusion) of platelets is generally for more severe thrombocytopenia, such as defined by a platelet count below a certain threshold (e.g., less than about 20,000/μL, less than about 10,000/μL, less than about 5,000/μL). Such transfusions may be prophylactic and/or therapeutic. Generally, in certain preferred embodiments for conditions requiring platelet transfusion, such as for example, patients undergoing chemotherapy or hematopoietic stem cell transplantation, platelets are administered as provided herein when the platelet count is less than about 10,000/μL. Some conditions in which platelet administration is medically indicated may be in patients with elevated risk for a leukocyte-related transfusion complication. A variety of leukocyte-related transfusion complications (e.g., leukocyte-mediated adverse reactions) have been reported, including increased risk of immune mediated-complications, such as transfusion-associated graft versus host disease (TA-GVHD), alloimmunization (e.g., HLA-alloimmunization), platelet refractoriness, febrile non-hemolytic transfusion reactions, immunosuppression and transmission or reactivation of viruses, such as cytomegalovirus. Transfused donor leukocytes may lead to complications by proliferating and destroying susceptible tissues in an immunocompromised recipient, or even when non-proliferating, by triggering immune activities through displayed antigens (e.g., HLA markers) and/or stimulation or secretion of cytokines.

Non-limiting examples of patients with elevated risk for a leukocyte-related transfusion complication, include those with the following conditions: cytopenias from whole body radiation exposure, recipients of peripheral blood stem cell or bone marrow transplant, recipients of ablative chemotherapy and/or radiotherapy, immunodeficiency, congenital immunodeficiency (e.g., congenital immunodeficiency syndrome), hematopoietic transplantation, bone marrow transplantation (e.g., allogeneic, autologous), immunocompromised recipients of organ transplantation, massive blood loss of severe trauma, hematologic malignancy or solid tumor, such as for example, malignant lymphomas, leukemias and hematological malignancies (e.g., acute myeloid leukemia, acute lymphoblastic leukemia, acute promyelocytic leukemia, chronic myelogenous leukemia, chronic lymphocytic leukemia, Hodgkin lymphoma, Non-Hodgkin lymphoma, myeloma, myelodysplastic syndrome, aplastic anemia), other hematologic disorders, solid tumors treated with high-dose chemotherapy or radiation, and exchange transfusions.

In one aspect of the present disclosure, a method of providing a platelet product to a subject in need thereof is described, comprising administering to a human subject with elevated risk for a leukocyte-related transfusion complication a platelet product, wherein the platelet product comprises one or more platelet components (e.g., whole blood derived platelet components, platelet concentrates, platelet preparations) prepared from whole blood donation(s), and wherein the platelet product has been subjected (e.g., during preparation of the platelet product, after preparation of the platelet product) to photochemical treatment with a psoralen compound (e.g., psoralen derivative, salt thereof) to inactivate pathogens and leukocytes, if present, and wherein the platelet product has not been subjected (e.g., during preparation of the platelet product, after preparation of the platelet product) to treatment with either ionizing radiation (e.g., gamma irradiation, X-ray irradiation, X-irradiation) or a filtration medium to reduce the level of contaminating leukocytes. In some embodiments, the one or more platelet components (e.g., whole blood derived platelet components) are prepared from whole blood donation(s) by a buffy coat method. In some embodiments, the one or more platelet components (e.g., whole blood derived platelet components) are prepared from whole blood donation(s) by a platelet rich plasma (PRP) method. In some embodiments, the platelet product has been subjected to photochemical treatment with a psoralen compound after preparation of the platelet product. In some embodiments, the platelet product has been subjected to photochemical treatment with a psoralen compound during preparation of the platelet product (e.g., treatment of platelet component(s)). In some embodiments, the one or more platelet components of the platelet product have been subjected to photochemical treatment with a psoralen compound.

For example, the present disclosure describes a method of providing a platelet product to a subject in need thereof, comprising administering to a human subject with elevated risk for a leukocyte-related transfusion complication a platelet product, wherein the platelet product comprises one or more platelet components and the one or more platelet components are prepared from whole blood donation(s) by a buffy coat method or a platelet rich plasma (PRP) method, and wherein the platelet product has been subjected to photochemical treatment with a psoralen compound (e.g., to inactivate pathogens and leukocytes, if present), and wherein the platelet product has not been subjected to treatment with either ionizing radiation or a filtration medium to reduce the level of contaminating leukocytes. In some embodiments, the one or more platelet components are prepared by a buffy coat method. In some embodiments, the platelet product has been subjected to photochemical treatment with a psoralen compound. In some embodiments, the platelet product has been subjected to photochemical treatment with a psoralen compound during preparation of the platelet product. In some embodiments, the one or more platelet components of the platelet product have been subjected to photochemical treatment with a psoralen compound.

In another example, the present disclosure sets forth a method of providing a platelet product to a subject in need thereof, comprising administering to a human subject with elevated risk for a leukocyte-related transfusion complication a platelet product, wherein the platelet product comprises one or more whole blood-derived platelet components prepared from whole blood donation(s); wherein the platelet product or platelet component(s) therein have been subjected to photochemical treatment with a psoralen compound (e.g., to inactivate pathogens and leukocytes, if present); and wherein the platelet product or platelet component(s) therein have not been subjected to treatment with either ionizing radiation or a filtration medium to reduce the level of contaminating leukocytes. In some embodiments, the one or more platelet components are prepared by a buffy coat method. In some embodiments, the one or more platelet components are prepared by a PRP method.

In another aspect of the present disclosure, methods are provided for preparing a transfusion ready platelet product for administration to a subject (e.g., subject with elevated risk for a leukocyte-related transfusion complication) in need thereof, comprising: a) preparing from whole blood donation(s) one or more platelet components (e.g., whole blood-derived platelet components, isolated platelet components, platelet concentrates, platelet preparations); b) subjecting the one or more platelet components to photochemical treatment with a psoralen compound (e.g., to inactivate pathogens and leukocytes, if present), comprising adding to the one or more platelet components the psoralen compound and exposing the mixture of platelet component(s) and psoralen compound to ultraviolet A light; and c) transferring the photochemical treated platelet component(s) to one or more storage containers with suitable labeling for human use, to provide a transfusion ready platelet product. In some embodiments, transferring the photochemical treated platelet component(s) to one or more storage containers further comprises combining two or more photochemical treated platelet components. In some embodiments, the method further comprises d) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration). In some embodiments, the method further comprises administering (e.g. infusing) the transfusion ready platelet product to a subject in need thereof.

In some aspects, a method of preparing a transfusion ready platelet product for administration to a subject (e.g., subject with elevated risk for a leukocyte-related transfusion complication) in need thereof is provided, comprising:
a) preparing from whole blood donation(s) one or more platelet components (e.g., whole blood-derived platelet components, isolated platelet components, platelet concentrates, platelet preparations);
b) subjecting the one or more platelet components to photochemical treatment with a psoralen compound (e.g., to inactivate pathogens and leukocytes, if present), comprising adding to the one or more platelet components the psoralen compound and exposing the mixture of platelet component(s) and psoralen compound to ultraviolet A light;
c) transferring the photochemical treated platelet component(s) to one or more storage containers with suitable labeling for human use, to provide a transfusion ready platelet product; and
d) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration).

In some embodiments, the subject in need thereof is a subject with elevated risk for a leukocyte-related transfusion complication. The one or more platelet components may be prepared, for example, by a buffy coat method or a platelet rich plasma (PRP) method. In some embodiments, the whole blood donation(s) and/or the platelet components have not been subjected to treatment with ionizing radiation (e.g., gamma irradiation, X-ray irradiation, X-irradiation) or a filtration media (e.g., leukofiltration, leukocyte filter) to reduce the level of contaminating leukocytes. Preferably the whole blood donation(s) and the platelet components have not been subjected to treatment with ionizing radiation or a filtration media. Further, the one or more storage containers with suitable labeling for human use may comprise labeling indicating that treatment with ionizing radiation (e.g., gamma irradiation, X-ray irradiation, X-irradiation) or a filtration medium (e.g., leukocyte filter) is not required prior to administration of the platelet product to a subject (e.g., human subject). Alternatively or in addition, the one or more storage containers with suitable labeling for human use may include instructions indicating that treatment with ionizing radiation (e.g., gamma irradiation, X-ray irradiation, X-irradiation) or a filtration medium (e.g., leukocyte filter) is not required prior to administration of the platelet product to a subject (e.g., human subject). Alternatively or in addition, the labeling and/or instructions may indicate that the transfusion ready platelet product has not been subjected to treatment with either ionizing radiation (e.g., gamma irradiation, X-ray irradiation, X-irradiation) or a filtration medium (e.g., leukocyte reduction filter) to reduce the level of contaminating leukocytes.

In some embodiments, the disclosure provides a method of preparing a transfusion ready platelet product for administration to a subject in need thereof with elevated risk for a leukocyte-related transfusion complication, comprising: a) preparing from whole blood donations by a buffy coat method or a PRP method one or more platelet components; b) subjecting the one or more platelet components to photochemical treatment with a psoralen compound to inactivate pathogens and leukocytes, if present, comprising adding to the one or more platelet components the psoralen compound and exposing the mixture of platelet component(s) and psoralen compound to ultraviolet A light; and c) transferring the photochemical treated platelet component(s) to one or more storage containers with suitable labeling for human use, to provide a transfusion ready platelet product, wherein the one of more storage containers comprise labeling indicating that treatment with ionizing radiation or a filtration medium is not required prior to administration of the platelet product to a human subject. In some embodiments, the method further comprises d) providing the transfusion ready platelet product for administration to a human subject in need thereof, without a further step of treatment with either gamma radiation or a leukocyte reduction filter.

In another aspect of the present disclosure, methods are provided for preparing a transfusion ready platelet product for administration to a subject (e.g., subject with elevated risk for a leukocyte-related transfusion complication) in need thereof, comprising: a) preparing from whole blood donations a plurality of platelet components (e.g., whole blood-derived platelet components, isolated platelet components, platelet concentrates, platelet preparations); b) pooling two or more of the platelet components to produce a pooled platelet product; c) subjecting the pooled platelet product or each platelet component (e.g., prior to pooling) to photochemical treatment with a psoralen compound (e.g., to inactivate pathogens and leukocytes, if present), comprising adding to the pooled platelet product or each platelet component the psoralen compound and exposing the mixture of pooled platelet product or platelet component and psoralen compound to ultraviolet A light; and d) transferring the photochemical treated pooled platelet product to a storage container to provide a transfusion ready platelet product. In some embodiments, the method further comprises e) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration). In some embodiments, the method further comprises administering (e.g. infusing) the transfusion ready platelet product to a subject in need thereof.

In some aspects, a method of preparing a transfusion ready platelet product for administration to a subject in need thereof is provided, comprising (e.g., comprising the steps of):
a) preparing from whole blood donations a plurality of platelet components (e.g., whole blood-derived platelet components, isolated platelet components, platelet concentrates, platelet preparations);
b) pooling two or more of the platelet components to produce a pooled platelet product;
c) subjecting the pooled platelet product or each platelet component (e.g., prior to pooling) to photochemical treatment with a psoralen compound to inactivate pathogens and leukocytes, if present, comprising adding to the pooled platelet product or each platelet component the psoralen compound and exposing the mixture of pooled platelet product or platelet component and psoralen compound to ultraviolet A light;
d) transferring the photochemical treated pooled platelet product to a storage container to provide a transfusion ready platelet product (e.g., for administration to a human subject in need thereof); and
e) providing the transfusion ready platelet product for administration to a human subject in need thereof, without a further step of treatment with either ionizing radiation (e.g., gamma irradiation, X-ray irradiation, X-irradiation) or a filtration medium (e.g., leukofiltration) to reduce the level of contaminating leukocytes.

In some embodiments, the subject in need thereof is a subject with elevated risk for a leukocyte-related transfusion complication. The plurality of platelet components may be prepared, for example, by a buffy coat method or a platelet rich plasma (PRP) method. Further, the platelet components may be subjected to photochemical treatment with a psoralen compound, for example, prior to pooling or after pooling (e.g., pooled platelet product). In some embodiments, the whole blood donation(s) and/or the platelet components have not been subjected to treatment with ionizing radiation (e.g., gamma irradiation, X-ray irradiation, X-irradiation) or a filtration media (e.g., leukofiltration, leukocyte filter, leukocyte reduction filter) to reduce the level of contaminating leukocytes. Preferably the whole blood donation(s) and the platelet components have not been subjected to treatment with ionizing radiation or a filtration media.

In some embodiments, the disclosure provides a method of preparing a transfusion ready platelet product for administration to a subject in need thereof with elevated risk for a leukocyte-related transfusion complication, comprising: a) preparing from whole blood donations by a buffy coat method or a PRP method a plurality of platelet components; b) pooling two or more of the platelet components to produce a pooled platelet product; c) subjecting the pooled platelet product or each platelet component to photochemical treatment with a psoralen compound to inactivate pathogens and leukocytes, if present, comprising adding to the pooled platelet product or each platelet component the psoralen compound and exposing the mixture of pooled platelet product or platelet component and psoralen compound to ultraviolet A light; and d) transferring the photochemical treated pooled platelet product to a storage container to provide a transfusion ready platelet product, wherein the storage container comprises labeling indicating that treatment with ionizing radiation or a filtration medium is not required prior to administration of the platelet product to a human subject. In some embodiments, the method further comprises d) providing the transfusion ready platelet product for administration to a human subject in need thereof, without a further step of treatment with either gamma radiation or a leukocyte reduction filter.

For example, a method of preparing a transfusion ready platelet product for administration to a subject in need thereof is provided, comprising:
a) preparing from whole blood donations a plurality of platelet components by a buffy coat or platelet rich plasma (PRP) method;
b) pooling two or more of the platelet components prepared by the buffy coat method or two or more of the platelet components prepared by the PRP method, to produce a pooled platelet product;
c) subjecting the pooled platelet product or each platelet component to photochemical treatment with a psoralen compound to inactivate pathogens and leukocytes, if present, comprising adding to the pooled platelet product or each platelet component the psoralen compound and exposing the mixture of pooled platelet product or platelet component and psoralen compound to ultraviolet A light;
d) transferring the photochemical treated pooled platelet product to a storage container to provide a transfusion ready platelet product; and
e) providing the transfusion ready platelet product for administration to a human subject in need thereof, without a further step of treatment with either ionizing radiation or a filtration medium to reduce the level of contaminating leukocytes.

In another example, a method of preparing a transfusion ready platelet product for administration to a subject in need thereof, comprising:
a) preparing from whole blood donations a plurality of platelet components by a buffy coat or PRP method;
b) subjecting each platelet component to photochemical treatment with a psoralen compound to inactivate pathogens and leukocytes, if present, comprising adding to each platelet component the psoralen compound and exposing the mixture of platelet component and psoralen compound to ultraviolet A light;
c) pooling two or more of the platelet components prepared by the buffy coat method or two or more of the platelet components prepared by the PRP method, to produce a pooled platelet product;
d) transferring the photochemical treated pooled platelet product to a storage container to provide a transfusion ready platelet product; and
e) providing the transfusion ready platelet product for administration to a human subject in need thereof, without a further step of treatment with either ionizing radiation or a filtration medium to reduce the level of contaminating leukocytes.

A transfusion ready platelet product as provided herein may be suitable for administration to a human subject with elevated risk for a leukocyte-related transfusion complication. For example, a method of preparing a transfusion ready platelet product for administration to a subject in need thereof is provided, comprising the steps of:
a) preparing from whole blood donations a plurality of platelet components by a buffy coat or PRP method;
b) subjecting each platelet component to photochemical treatment with a psoralen compound to inactivate pathogens and leukocytes, if present, comprising adding to each platelet component the psoralen compound and exposing the mixture of platelet component and psoralen compound to ultraviolet A light;
c) pooling two or more of the platelet components prepared by the buffy coat method or two or more of the platelet components prepared by the PRP method, to produce a pooled platelet product;
d) transferring the photochemical treated pooled platelet product to a storage container to provide a transfusion ready platelet product; and
e) providing the transfusion ready platelet product for administration to a human subject in need thereof with elevated risk for a leukocyte-related transfusion complication, without a further step of treatment with either ionizing radiation or a filtration medium to reduce the level of contaminating leukocytes.

In another example, a method of preparing a transfusion ready platelet product for administration to a subject in need thereof is provided, comprising:
a) preparing from whole blood donations a plurality of platelet components by a buffy coat or PRP method;
b) pooling two or more of the platelet components prepared by the buffy coat method or two or more of the platelet components prepared by the PRP method, to produce a pooled platelet product;
c) subjecting the pooled platelet product to photochemical treatment with a psoralen compound to inactivate pathogens and leukocytes, if present, comprising adding to the pooled platelet product the psoralen compound and exposing the mixture of pooled platelet product and psoralen compound to ultraviolet A light;
d) transferring the photochemical treated pooled platelet product to a storage container to provide a transfusion ready platelet product; and
e) providing the transfusion ready platelet product for administration to a human subject in need thereof, without a further step of treatment with either ionizing radiation or a filtration medium to reduce the level of contaminating leukocytes.

A transfusion ready platelet product as provided herein may be suitable for administration to a human subject with elevated risk for a leukocyte-related transfusion complication. For example, a method of preparing a transfusion ready platelet product for administration to a subject in need thereof is provided, comprising the steps of:
a) preparing from whole blood donations a plurality of platelet components by a buffy coat or PRP method;
b) pooling two or more of the platelet components prepared by the buffy coat method or two or more of the platelet components prepared by the PRP method, to produce a pooled platelet product;
c) subjecting the pooled platelet product to photochemical treatment with a psoralen compound to inactivate pathogens and leukocytes, if present, comprising adding to the pooled platelet product the psoralen compound and exposing the mixture of pooled platelet product and psoralen compound to ultraviolet A light;
d) transferring the photochemical treated pooled platelet product to a storage container to provide a transfusion ready platelet product; and
e) providing the transfusion ready platelet product for administration to a human subject in need thereof with elevated risk for a leukocyte-related transfusion complication, without a further step of treatment with either ionizing radiation or a filtration medium to reduce the level of contaminating leukocytes

In some embodiments, a transfusion ready platelet product as provided herein reduces leukocyte-related transfusion complications (e.g., the risk of leukocyte-related transfusion complications) in a recipient of a platelet product (e.g., the transfusion ready platelet product). In some embodiments, a transfusion ready platelet product as provided herein may comprise a platelet product in a storage container with suitable labeling for human use, wherein the labeling indicates that treatment with ionizing radiation (e.g., gamma irradiation, X-ray irradiation, X-irradiation) or a filtration medium (e.g., leukocyte filter) is not required prior to administration of the platelet product to a human subject. Alternatively or in addition, a transfusion ready platelet product as provided herein may comprise a platelet product in a storage container and instructions for human use, wherein the instructions indicate that treatment with ionizing radiation (e.g., gamma irradiation, X-ray irradiation, X-irradiation) or a filtration medium (e.g., leukocyte filter) is not required prior to administration of the platelet product to a human subject. Alternatively or in addition, the labeling and/or instructions may indicate that the transfusion ready platelet product has not been subjected to treatment with either ionizing radiation (e.g., gamma irradiation. X-ray irradiation, X-irradiation) or a filtration medium to reduce the level of contaminating leukocytes.

In another aspect of the present disclosure, a method of treating a subject in need thereof with a platelet product is provided, comprising administering to a human subject a transfusion ready platelet product prepared according to any of the aforementioned methods. In some embodiments, the subject is a subject with elevated risk for a leukocyte-related transfusion complication. In some embodiments, the method reduces a leukocyte-related transfusion complication (e.g., after administration of the platelet product to a subject). In some embodiments, the leukocyte-related transfusion complication is transfusion-associated graft-versus-host disease (TA-GVHD). In some embodiments, the leukocyte-related transfusion complication is alloimmunization. In some embodiments, the leukocyte-related transfusion complication is alloimmune platelet refractoriness. In some embodiments, the leukocyte-related transfusion complication is cytokine stimulation and/or production. In some embodiments, the leukocyte-related transfusion complication is microchimerism. In some embodiments, the leukocyte-related transfusion complication is a febrile non-hemolytic transfusion reaction (FNHTR).

In another aspect of the present disclosure, a method for reducing leukocyte-related transfusion complications in a recipient of a platelet product is provided, comprising:
a) preparing from whole blood donations a plurality of platelet components (e.g., whole blood-derived platelet components);
b) pooling two or more platelet components to produce a pooled platelet concentrate;
c) subjecting the pooled platelet product or each platelet component (e.g., prior to pooling) to photochemical treatment with a psoralen compound to inactivate pathogens and leukocytes, if present, comprising adding to the pooled platelet product or each platelet component the psoralen compound and exposing the mixture of pooled platelet product and psoralen compound or platelet component and psoralen compound to ultraviolet light;
d) transferring the photochemical treated pooled platelet product to a storage container to provide a transfusion ready platelet product; and
e) administering the transfusion ready platelet product to a human subject in need thereof, without a further step of treatment with either ionizing radiation (e.g., gamma irradiation, X-ray irradiation, X-irradiation) or a filtration medium (e.g., leukofiltration) to reduce the level of contaminating leukocytes.

The plurality of platelet components may be prepared, for example, by a buffy coat method or a platelet rich plasma (PRP) method. The platelet components may be subjected to photochemical treatment with a psoralen compound, for example, prior to pooling or after pooling (e.g., pooled platelet product). In some embodiments, the whole blood donation(s) and/or platelet components have not been subjected to treatment with ionizing radiation (e.g., gamma irradiation, X-ray irradiation, X-irradiation) or a filtration media (e.g., leukofiltration) to reduce the level of contaminating leukocytes. Preferably the whole blood donation(s) and the platelet components have not been subjected to treatment with ionizing radiation or a filtration media.

For example, a method for reducing leukocyte-related transfusion complications in a recipient of a platelet product is provided, comprising:
a) preparing from whole blood donations a plurality of platelet components by a buffy coat or PRP method;
b) pooling two or more platelet components prepared by the buffy coat method or two or more platelet components prepared by the PRP method to produce a pooled platelet concentrate;
c) subjecting the pooled platelet product or each platelet component to photochemical treatment with a psoralen compound to inactivate pathogens and leukocytes, if present, comprising adding to the pooled platelet product or each platelet component the psoralen compound and exposing the mixture of pooled platelet product or platelet component and psoralen compound to ultraviolet light;
d) transferring the photochemical treated pooled platelet product to a storage container to provide a transfusion ready platelet product; and
e) administering the transfusion ready platelet product to a human subject in need thereof, without a further step of treatment with either ionizing radiation or a filtration medium to reduce the level of contaminating leukocytes.

In some embodiments, the method comprises:
a) preparing from whole blood donations a plurality of platelet components by a buffy coat or PRP method;
b) subjecting each platelet component to photochemical treatment with a psoralen compound to inactivate pathogens and leukocytes, if present, comprising adding to each platelet component the psoralen compound and exposing the mixture of platelet component and psoralen compound to ultraviolet A light;
c) pooling two or more of the platelet components prepared by the buffy coat method or two or more of the platelet components prepared by the PRP method, to produce a pooled platelet product;
d) transferring the photochemical treated pooled platelet product to a storage container to provide a transfusion ready platelet product; and
e) administering the transfusion ready platelet product to a human subject in need thereof, without a further step of treatment with either ionizing radiation or a filtration medium to reduce the level of contaminating leukocytes.

In some embodiments, the method comprises:
a) preparing from whole blood donations a plurality of platelet components by a buffy coat or PRP method;
b) pooling two or more of the platelet components prepared by the buffy coat method or two or more of the platelet components prepared by the PRP method, to produce a pooled platelet product;
c) subjecting the pooled platelet product to photochemical treatment with a psoralen compound to inactivate pathogens and leukocytes, if present, comprising adding to the pooled platelet product the psoralen compound and exposing the mixture of pooled platelet product and psoralen compound to ultraviolet A light;
d) transferring the photochemical treated pooled platelet product to a storage container to provide a transfusion ready platelet product; and
e) administering the transfusion ready platelet product to a human subject in need thereof, without a further step of treatment with either ionizing radiation or a filtration medium to reduce the level of contaminating leukocytes.

In some embodiments a transfusion ready platelet product a provided herein is suitable for administration to a human subject with elevated risk for a leukocyte-related transfusion complication. In some embodiments, the recipient of a transfusion ready platelet product as provided herein is a human subject with elevated risk for a leukocyte-related transfusion complication. In some embodiments, a transfusion ready platelet product as provided herein may comprise a platelet product in a storage container with suitable labeling for human use, wherein the labeling indicates that treatment with ionizing radiation (e.g., gamma irradiation, X-ray irradiation, X-irradiation) or a filtration medium (e.g., leukocyte filter) is not required prior to administration of the platelet product to a subject (e.g., human subject). Alternatively or in addition, a transfusion ready platelet product as provided herein may comprise a platelet product in a storage container and instructions for human use, wherein the instructions indicate that treatment with ionizing radiation (e.g., gamma irradiation, X-ray irradiation, X-irradiation) or a filtration medium (e.g., leukocyte filter) is not required prior to administration of the platelet product to a subject (e.g., human subject). Alternatively or in addition, the labeling and/or instructions may indicate that the transfusion ready platelet product has not been subjected to treatment with either ionizing radiation (e.g., gamma irradiation, X-ray irradiation, X-irradiation) or a filtration medium to reduce the level of contaminating leukocytes.

In another aspect of the present disclosure, a transfusion ready platelet product is provided, comprising one or more (e.g., a plurality of) whole blood-derived platelet components in a storage container with suitable labeling for human use, wherein the one or more platelet component(s) have been subjected to photochemical treatment with a psoralen compound (e.g., to inactivate pathogens and leukocytes, if present), and wherein the one or more platelet component(s) have not been subjected to treatment with ionizing radiation (e.g., gamma irradiation, X-ray irradiation, X-irradiation) or a filtration media (e.g., leukofiltration, leukocyte reduction filter) (e.g., to reduce the level of contaminating leukocytes), wherein the transfusion ready platelet product comprises suitable labeling and/or accompanying instructions indicating that treatment (e.g., a further step of treatment) with either ionizing radiation (e.g., gamma irradiation, X-ray irradiation, X-irradiation) or a filtration medium (e.g., leukofiltration, leukocyte reduction filter) is not required for administration to a subject. In some embodiments, the transfusion ready platelet product further comprises suitable labeling and/or accompanying instructions indicating that the transfusion ready platelet product has not been subjected to treatment with either ionizing radiation (e.g., gamma irradiation, X-ray irradiation, X-irradiation) or a filtration medium (e.g., leukofiltration, leukocyte reduction filter). The one or more whole blood-derived platelet components are prepared, for example, by a buffy coat method or a platelet rich plasma (PRP) method. In some embodiment, the transfusion ready platelet product comprises 4-6 platelet components. In some embodiments, the transfusion ready platelet product comprises at least about 2.5×1011, at least about 2.6×1011, at least about 2.7×1011, at least about 2.8×1011, at least about 2.9×1011, at least about 3.0×1011, at least about 3.5×1011, or at least about 4.0×1011 or more platelets. In some embodiments, the transfusion ready platelet product is a platelet product for use in treating a subject in need thereof. In some embodiments, the transfusion ready platelet product is a platelet product for use in treating a subject with elevated risk for a leukocyte-related transfusion complication. In some embodiments, the transfusion ready platelet product is a platelet product for use in reducing leukocyte-related transfusion complications in a recipient of a platelet product.

In some embodiments, the disclosure provides a transfusion ready platelet product, comprising, in a storage container with suitable labeling for human use, one or more platelet components prepared by a buffy coat method or a PRP method, wherein the one or more platelet components have been subjected to photochemical treatment with a psoralen compound to inactivate pathogens and leukocytes, if present, and wherein the one or more platelet components have not been subjected to treatment with gamma irradiation or a leukocyte reduction filter, and wherein the transfusion ready platelet product comprises suitable labeling and/or accompanying instructions indicating that a further step of treatment with either gamma irradiation or leukocyte reduction filter is not required for administration to a subject. In some embodiments, the disclosure provides a transfusion ready platelet product, comprising, in a storage container with suitable labeling for human use, a mixture of 4-6 platelet components prepared by a buffy coat method or a PRP method, wherein the platelet components have been subjected to photochemical treatment with a psoralen compound to inactivate pathogens and leukocytes, if present, and wherein the platelet components have not been subjected to treatment with gamma irradiation or a leukocyte reduction filter, and wherein the transfusion ready platelet product comprises suitable labeling and/or accompanying instructions indicating that a further step of treatment with either gamma irradiation or leukocyte reduction filter is not required for administration to a subject. In some embodiments, the transfusion ready platelet product further comprises suitable labeling and/or accompanying instructions indicating that the transfusion ready platelet product has not been subjected to treatment with either gamma irradiation or a leukocyte reduction filter. In some embodiments, the transfusion ready platelet product is a platelet product for use in treating a subject in need thereof. In some embodiments, the transfusion ready platelet product is a platelet product for use in treating a subject with elevated risk for a leukocyte-related transfusion complication. In some embodiments, the transfusion ready platelet product is a platelet product for use in reducing leukocyte-related transfusion complications in a recipient of a platelet product.

In another aspect, the present disclosure sets forth a method of treating a subject in need thereof with a platelet product, comprising administering to a human subject a transfusion ready platelet product as provided herein. In another aspect, the present disclosure sets forth a method of providing a platelet product to a subject in need thereof, comprising administering (e.g., infusing) to a human subject a transfusion ready platelet product as provided herein. In some embodiments, the subject is a subject with elevated risk for a leukocyte-related transfusion complication. In some embodiments, the method comprises administering the transfusion ready platelet product 2 or more times, 3 or more times, 4 or more times or 5 or more times In some embodiments, the subject is a human subject with elevated risk for a leukocyte-related transfusion complication. In some embodiments, the method comprises administering the transfusion ready platelet product to the subject 2 or more times. In some embodiments, the method comprises administering the transfusion ready platelet product to the subject 3 or more times, 4 or more times or 5 or more times. In some embodiments, the method comprises administering two or more transfusions (e.g., units, doses) of the transfusion ready platelet product to the subject. In some embodiments, the method comprises administering three or more transfusions, 4 or more transfusions or 5 or more transfusions of the transfusion ready platelet product to the subject.

The efficacy outcome of platelet transfusion may be determined by any of a variety of known methods (e.g., standard clinical measure), such as for example, 1-hour platelet count increment (CI) and 1-hour corrected count increment (CCI). CCI may be calculated as the difference between the platelet count within one hour after transfusion and the platelet count before transfusion, multiplied by the body-surface area (m2) and divided by platelet dose transfused (×10-11). A satisfactory response may be defined, for example, as a CCI of ≥5,000. The CI calculation does not consider body-surface area. Additional measures may include, for example, the CI and/or CCI 24 hours after platelet transfusion, the number of transfusions during the period of platelet support, the interval between platelet transfusions, clinical hemostasis before and after platelet transfusion, the proportion of patients with refractoriness to platelet transfusion (e.g., defined as 2 successive 1-hour CCIs<5000), the proportion of patients with alloimmunization (e.g., defined as serologic conversion of the lymphocytotoxicity assay).

The invention is illustrated further by the following examples, which are not to be construed as limiting the invention in scope or spirit to the specific procedures described in them.

### EXAMPLES

### Example 1: Transfusion of Pathogen Inactivated Platelets without Gamma Irradiation or Leukofiltration to HSCT Patients

In a clinical study, platelets prepared by pathogen inactivation using a psoralen (amotosalen)-UVA based photochemical treatment (INTERCEPT™ Blood System, Cerus Corp., Concord, Calif.), but with no additional treatment with gamma irradiation or leukofiltration step to inactivate or remove leukocytes, were evaluated in HSCT patients. Specifically, INTERCEPT-treated platelet products (designated Treated Platelets) were prepared using the buffy coat method, as pools of five ABO matched, non-leukofiltered platelet components in platelet additive solution (PAS), using standard buffy coat procedures for the blood bank and INTERCEPT pathogen inactivation as recommended by the manufacturer. Platelet units were administered to patients undergoing autologous HSCT or identical ABO blood group allogenic HSCT. An additional group of patients undergoing autologous HSCT or identical ABO blood group allogenic HSCT received conventional gamma irradiated platelets prepared by the PRP method, as control (designated Control Platelets). Each patient could receive up to 5 units of either treated or control platelets in this study. A total of 36 transfusions in 13 patients were performed with the Treated Platelet units and 88 transfusions in 31 patients were performed with the Control Platelet units, and the patient profiles were as illustrated in Table 2 below.

TABLE 2
Patient and transplant characteristics

| | Treated platelets | Control Platelets |
|---|---|---|
| Gender | 8 male, | 11 male, |
| | 5 female | 20 female |
| Diagnosis: | | |
| Acute myeloid leukemia | 9 | 13 |
| Acute lymphoblastic leukemia | 2 | 7 |
| Chronic myeloid leukemia | 1 | 1 |
| Multiple myeloma | 1 | 1 |
| Chronic lymphocytic leukemia | 1 | — |
| Myelodysplastic syndrome | 2 | 2 |
| Myeloproliferative neoplasm | 2 | 2 |
| Non-Hodgkin's lymphoma | 5 | 5 |
| Graft Type: | | |
| Bone marrow | 9 | 15 |
| Peripheral blood stem cell | 4 | 16 |
| Blood Group (A/B/O/AB) | 3/4/5/1 | 6/13/11/1 |
| Transplant characteristics: | | |
| Autologous | 2 | 5 |
| Allogeneic | 11 | 26 |
| Allo-donors (related/unrelated) | 6/5 | 17/9 |

The primary efficacy endpoint for the platelet transfusion was the 1 hour corrected count increment (CCI) and the primary safety endpoint was the proportion of patients who experienced an adverse reaction following transfusion of the treated platelets. As shown in Table 3 below, transfusion of the INTERCEPT treated platelets achieved adequate platelet count increments. Importantly, no leukocyte-related transfusion complications were observed with the Treated Platelets during the study period. Three patients in the Control group suffered complications, one with refractory disease post HSCT, one with hemorrhagic cystitis and one with CMV reactivation followed by graft failure, gut GVHD and post-transplant lymphoproliferative disease.

TABLE 3
Mean values for platelet (PLT) counts (×10⁹/L) and CCI

| | Treated Platelets | | Control Platelets | |
|---|---|---|---|---|
| Pre-transfusion PLT count (median, range) | 10 | (2-49) | 9 | (2-42) |
| Post-transfusion PLT count (median, range) | 50.5 | (24-98) | 45 | (3-90) |
| Count increment (median, range) | 38.5 | (12-91) | 34.5 | (−3*-79) |
| 1-hour CCI (mean, SD) | 20288, 7886 | | 15662, 9329 | |
| 1-hour CCI (median, range) | 20479 (4139-40283) | | 16815 (−1380-42809) | |
| No. of transfusions with 1-hr CCI >5,000 | 35 of 36 | (97%) | 72 of 88 | (82%) |
| No. of transfusions with 1-hr CCI >10,000 | 35 of 36 | (97%) | 66 of 88 | (75%) |
| No. of transfusions with 1-hr CCI <5,000 | 1 of 36 | (3%) | 6 of 88 | (6.8%) |
| No. of transfusions with 1-hr CCI <10,000 | 1 of 36 | (3%) | 8 of 88 | (9%) |

*Three Control Platelet transfusions resulted in post-transfusion platelet counts lower than pre-transfusion platelet counts.

Additional data from the above study were collected for more subjects in the Treated Platelets group after further enrollment. At this analysis, a total of 60 transfusions in 28 patients were performed with the Treated Platelet units, and the patient profiles for this group, as well as the earlier enrolled Control group, were as illustrated in Table 4 below.

TABLE 4
Patient and transplant characteristics

| | Treated Platelets | Control Platelets |
|---|---|---|
| Gender | 8 male, | 11 male, |
| | 5 female | 20 female |
| Diagnosis: | | |
| Acute myeloid leukemia | 13 | 13 |
| Acute lymphoblastic leukemia | 6 | 7 |
| Chronic myeloid leukemia | 1 | 1 |
| Multiple myeloma | 2 | 1 |
| Chronic lymphocytic leukemia | 1 | — |
| Myelodysplastic syndrome | 2 | 2 |
| Myeloproliferative neoplasm | — | 2 |
| Non-Hodgkin's lymphoma | 1 | 5 |
| Hodgkin lymphoma | 1 | — |
| Severe Aplastic Anemia | 1 | — |
| Graft Type: | | |
| Bone marrow | 15 | 15 |
| Peripheral blood stem cell | 12 | 16 |
| BM + PBSC | 1 | — |
| Blood Group (A/B/O/AB) | 7/6/14/1 | 6/13/11/1 |
| Transplant characteristics: | | |
| Autologous | 5 | 5 |
| Allogeneic | 23 | 26 |
| Allo-donors (related/unrelated) | 16/7 | 17/9 |

For the primary efficacy endpoint, transfusion of the INTERCEPT treated platelets achieved adequate 1 hour corrected count increment (CCI) for platelet counts, with the mean for Treated group significantly higher than the Control group, as shown in Table 5 below. For the primary safety endpoint of proportion of patients who experienced an adverse reaction following transfusion of the treated platelets, no leukocyte-related transfusion complications were observed with the Treated Platelets during the study period.

TABLE 5
Mean values for platelet (PLT) counts (×10⁹/L) and CCI

| | Treated Platelets | | Control Platelets | |
|---|---|---|---|---|
| Pre-transfusion PLT count (median, range) | 10 | (2-49) | 9 | (2-42) |
| Post-transfusion PLT count (median, range) | 49.5 | (19-123) | 46 | (3-90) |
| Count increment (median, range) | 38 | (4-113) | 34.5 | (−3*-79) |
| 1-hour CCI (mean, SD) | 18974 | (9466) | 15662 | (9329) |
| 1-hour CCI (median, range) | 16972 (2181-40888) | | 16816 (−1380-42809) | |
| No. of transfusions with 1-hr CCI >5,000 | 56 of 60 | (93%) | 72 of 88 | (82%) |
| No. of transfusions with 1-hr CCI >10,000 | 50 of 60 | (83%) | 66 of 88 | (75%) |
| No. of transfusions with 1-hr CCI <5,000 | 4 of 60 | (6.7%) | 6 of 88 | (6.8%) |
| No. of transfusions with 1-hr CCI <10,000 | 7 of 60 | (11.6%) | 8 of 88 | (9%) |

*Three Control Platelet transfusions resulted in post-transfusion platelet counts lower than pre-transfusion platelet counts.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Wherever an open-ended term is used to describe a feature or element, it is specifically contemplated that a closed-ended term can be used in place of the open-ended term without departing from the spirit and scope of the disclosure. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the description and does not pose a limitation on the scope of the description unless otherwise claimed. No language in the specification should be construed as indicating any non-claimed element as essential to the practice of the compositions and methods disclosed herein.

Preferred embodiments are described herein. Variations of those preferred embodiments may become apparent to those working in the art upon reading the foregoing description. It is expected that skilled artisans will be able to employ such variations as appropriate, and practice the compositions and methods disclosed herein otherwise than as specifically described herein. Accordingly, the compositions and methods disclosed herein include all modifications and equivalents of the subject matter recited in the claims appended hereto as permitted by applicable law. Moreover, any combination of the above-described elements in all possible variations thereof is encompassed by the description unless otherwise indicated herein or otherwise clearly contradicted by context.

1. A method of providing a platelet product to a subject in need thereof, comprising administering to a human subject with elevated risk for a leukocyte-related transfusion complication a platelet product,
wherein the platelet product comprises one or more whole blood-derived platelet components prepared from whole blood donation(s);
wherein the platelet product has been subjected to photochemical treatment with a psoralen compound to inactivate pathogens and leukocytes, if present; and
wherein the platelet product has not been subjected to treatment with either ionizing irradiation or a filtration medium to reduce the level of contaminating leukocytes.

2. The method of claim 1, wherein the one or more platelet components are prepared from whole blood donation(s) by a buffy coat method, or wherein the one or more platelet components are prepared from whole blood donation(s) by a platelet rich plasma (PRP) method.

3. (canceled)

4. The method of claim 1, wherein the platelet product is a pooled platelet product, comprising a mixture of at least two platelet components.

5. (canceled)

6. The method of claim 1, wherein the platelet product comprises about 2.0×10¹¹ to about 8.0×10¹¹ platelets.

7-10. (canceled)

11. The method of claim 1, wherein the platelet product has not been subjected to treatment with a leukocyte reduction filter.

12. The method of claim 1, wherein the platelet product is a transfusion ready platelet product.

13. The method of claim 1, wherein the subject with elevated risk for a leukocyte-related transfusion complication is a recipient of peripheral blood stem cell or bone marrow transplant.

14. The method of claim 1, wherein the subject with elevated risk for a leukocyte-related transfusion complication is a recipient of ablative chemotherapy and/or radiotherapy.

15. The method of claim 1, wherein the subject with elevated risk for a leukocyte-related transfusion complication is a subject diagnosed with a hematologic malignancy or solid tumor.

16. The method of claim 1, wherein the subject with elevated risk for a leukocyte-related transfusion complication is a subject diagnosed with an immunodeficiency.

17-19. (canceled)

20. The method of claim 1, wherein administering the platelet product to the subject results in a 1 hour corrected count increment (CCI) of greater than 5,000.

21. The method of claim 4, wherein the method reduces a leukocyte-related transfusion complication.

22. A method of preparing a transfusion ready platelet product for administration to a subject in need thereof, comprising:
a) preparing from whole blood donations a plurality of whole blood-derived platelet components;
b) pooling two or more of the platelet components to produce a pooled platelet product;
c) subjecting the pooled platelet product or each platelet component to photochemical treatment with a psoralen compound to inactivate pathogens and leukocytes, if present, comprising adding to the pooled platelet product or each platelet component the psoralen compound and exposing the mixture of pooled platelet product and psoralen compound or each platelet component and psoralen compound to ultraviolet A light; and
d) transferring the photochemical treated pooled platelet product to a storage container to provide a transfusion ready platelet product.

23. The method of claim 22, wherein the storage container comprises suitable labeling for human use.

24. The method of claim 22, wherein the subject with elevated risk for a leukocyte-related transfusion complication is a recipient of a peripheral blood stem cell or bone marrow transplant.

25. The method of claim 22, wherein the subject with elevated risk for a leukocyte-related transfusion complication is a recipient of ablative chemotherapy and/or radiotherapy.

26. The method of claim 22, wherein the subject with elevated risk for a leukocyte-related transfusion complication is a subject diagnosed with a hematologic malignancy or solid tumor.

27. The method of claim 22, wherein the subject with elevated risk for a leukocyte-related transfusion complication is a subject diagnosed with an immunodeficiency.

28. The method of claim 22, wherein the plurality of whole-blood derived platelet components is prepared from whole blood donations by a buffy coat method, or wherein the plurality of whole-blood derived platelet components is prepared from whole blood donations by a platelet rich plasma (PRP) method.

29-30. (canceled)

31. The method of claim 22, wherein the transfusion ready platelet product comprises about 2.0×10¹¹ to about 8.0×10¹¹ platelets.

32-35. (canceled)

36. The method of claim 22, wherein the transfusion ready platelet product has not been subjected to treatment with a leukocyte reduction filter.

37. The method of claim 22, wherein the transfusion ready platelet product has not been subjected to treatment with ionizing radiation.

38. The method of claim 22, wherein the storage container comprises labeling indicating that treatment with ionizing radiation or a leukocyte reduction filter is not required prior to administration of the platelet product to a subject.

39. The method of claim 22, wherein the storage container further comprises instructions indicating that treatment with ionizing radiation or a leukocyte reduction filter is not required prior to administration of the platelet product to a subject.

40. The method of claim 22, further comprising e) providing the transfusion ready platelet product for administration to a human subject in need thereof with elevated risk for a leukocyte-related transfusion complication, without a further step of treatment with either gamma irradiation or a filtration medium to reduce the level of contaminating leukocytes.

41. The method of claim 22, further comprising administering the transfusion ready platelet product to a subject in need thereof.

42. A method of providing a platelet product to a subject in need thereof, comprising administering to a human subject a transfusion ready platelet product prepared according to claim 22.

43. The method of claim 41, wherein the subject is a human subject with elevated risk for a leukocyte-related transfusion complication.

44-46. (canceled)

47. The method of claim 41, wherein administering the transfusion ready platelet product to the subject results in a 1 hour corrected count increment (CCI) of greater than 5,000.

48. The method of claim 7, wherein the method reduces a leukocyte-related transfusion complication.

49. A method for reducing leukocyte-related transfusion complications in a recipient of a platelet product, comprising:
a) preparing from whole blood donations a plurality of whole blood-derived platelet components;
b) pooling two or more platelet components to produce a pooled platelet concentrate;
c) subjecting the pooled platelet product or each platelet component to photochemical treatment with a psoralen compound to inactivate pathogens and leukocytes, if present, comprising adding to the pooled platelet product or each platelet component the psoralen compound and exposing the mixture of pooled platelet product and psoralen compound or platelet component and psoralen compound to ultraviolet light;
d) transferring the photochemical treated pooled platelet product to a storage container to provide a transfusion ready platelet product; and
e) administering the transfusion ready platelet product to a human subject in need thereof, without a further step of treatment with either gamma irradiation or a filtration medium to reduce the level of contaminating leukocytes.

50. The method of claim 49, wherein the storage container comprises suitable labeling for human use.

51. The method of claim 49, wherein the recipient is a human subject with elevated risk for a leukocyte-related transfusion complication.

52. The method of claim 49, wherein the subject with elevated risk for a leukocyte-related transfusion complication is a recipient of peripheral blood stem cell or bone marrow transplant.

53. The method of claim 49, wherein the subject with elevated risk for a leukocyte-related transfusion complication is a recipient of ablative chemotherapy and/or radiotherapy.

54. The method of claim 49, wherein the subject with elevated risk for a leukocyte-related transfusion complication is a subject diagnosed with a hematologic malignancy or solid tumor.

55. The method of claim 49, wherein the subject with elevated risk for a leukocyte-related transfusion complication is a subject diagnosed with an immunodeficiency.

56. The method of claim 49, wherein the plurality of platelet components is prepared by a buffy coat method, or wherein the plurality of platelet components is prepared by a PRP method.

57-58. (canceled)

59. The method of claim 49, wherein the transfusion ready platelet product comprises about 2.0×10¹¹ to about 8.0×10¹¹ platelets.

60-63. (canceled)

64. The method of claim 49, wherein the transfusion ready platelet product has not been subjected to treatment with a leukocyte reduction filter prior to administration to the subject.

65. The method of claim 9, wherein the transfusion ready platelet product has not been subjected to treatment with ionizing radiation.

66. The method of claim 49, wherein the storage container comprises labeling indicating that treatment with ionizing radiation or a leukocyte reduction filter is not required prior to administration of the platelet product to a subject.

67. The method of claim 49, wherein the storage container further comprises instructions indicating that treatment with ionizing radiation or a leukocyte reduction filter is not required prior to administration of the platelet product to a subject.

68-70. (canceled)

71. The method of claim 49, wherein administering the transfusion ready platelet product to the subject results in a 1 hour corrected count increment (CCI) of greater than 5,000.

72. A transfusion ready platelet product prepared by the method of claim 22.

73. A transfusion ready platelet product, comprising one or more whole blood-derived platelet components in a storage container with suitable labeling for human use, wherein the one or more platelet components have been subjected to photochemical treatment with a psoralen compound, and wherein the transfusion ready platelet product has not been subjected to treatment with ionizing radiation or a leukocyte reduction filter.

74. The transfusion ready platelet product of claim 73, wherein the storage container comprises labeling indicating that treatment with either ionizing radiation or a leukocyte reduction filter is not required for administration to a subject.

75. The transfusion ready platelet product of claim 73, wherein the one or more whole blood-derived platelet components are prepared by a buffy coat method, or wherein the one or more whole blood-derived platelet components are prepared by a platelet rich plasma (PRP) method.

76-77. (canceled)

78. The transfusion ready platelet product of claim 73, wherein the transfusion ready platelet product comprises about 2.0×10¹¹ to about 8.0×10¹¹ platelets.

79-82. (canceled)

83. The transfusion ready platelet product of claim 73, wherein the transfusion ready platelet product is a platelet product for use in treating a subject with elevated risk for a leukocyte-related transfusion complication.
